(19) United States
(12) Patent Application Publication
(10) Pub. No.: US 2023/0055382 A1
(43) Pub. Date: Feb. 23, 2023

(54) DETECTING GUT BARRIER DYSFUNCTION AND/OR CIRRHOSIS

(71) Applicant: MACFARLANE BURNET INSTITUTE FOR MEDICAL RESEARCH AND PUBLIC HEALTH LIMITED, Melbourne, Victoria (AU)

(72) Inventors: David Anderson, Melbourne (AU); Huy Van, Melbourne (AU); Jessica Howell, Melbourne (AU)

(21) Appl. No.: 17/794,750

(22) PCT Filed: Jan. 22, 2021
(86) PCT No.: PCT/AU2021/050037

§ 371 Date: Jul. 22, 2022

(30) Foreign Application Priority Data
Jan. 24, 2020 (AU) 2020900194

(51) Int. Cl. G01N 33/68; G01N 33/543

(52) U.S. Cl. CPC G01N 33/6893; G01N 33/54388; G01N 2800/085; G01N 2800/06

(57) ABSTRACT

The present invention relates to methods, kits and a test strip for detecting gut barrier dysfunction and/or cirrhosis in a subject. In addition, a method of treating a subject with gut barrier dysfunction and/or cirrhosis is provided.

### FIELD

The field of the specification is broadly detecting gut barrier dysfunction and/or cirrhosis in a subject. More specifically, methods, kits and test strips for identifying and monitoring gut barrier dysfunction and/or cirrhosis in a subject. In addition, a method of treating a subject with gut barrier dysfunction and/or cirrhosis is provided.

### BACKGROUND

Bibliographic details of references in the subject specification are also listed at the end of the specification.

Reference to any prior art in this specification is not, and should not be taken as, acknowledgement or any form of suggestion that this prior art forms part of the common general knowledge in any country.

Gut barrier dysfunction, also known as “increased intestinal permeability” or as “gut leakage”, refers to a disturbed state of a patients' intestinal barrier function, allowing penetration of harmful materials including viable microorganisms or their by-products such as toxic lipopolysaccharide (LPS) and antigens that are normally excluded from the circulation. This in turn leads to chronic immune activation (either systemic as seen in HIV infection, or local as seen in inflammatory bowel disease), which is recognized as a primary driver of pathogenesis in many conditions, and a suspected role in many others. Pathological conditions associated with gut barrier dysfunction can mostly be grouped into three types: 1) Intestinal barrier dysfunction observed in post-surgery patients subjected to major operations for diverse reasons; 2) Critically ill patients hospitalized in ICU, due to being severely injured, suffering burns, or with sepsis. Increased intestinal permeability is associated with the development of systemic inflammatory response and Multiple Organ Dysfunction Syndrome (MODS) in these patients; and 3) Patients with chronic pathologic conditions where gut barrier dysfunction leads to chronic immune activation associates with disease progression and/or development of complications and comorbidities from other organs. This group of gut leakage patients includes patients with liver cirrhosis, HIV infection, chronic viral hepatitis B or C, non-alcoholic steatohepatitis or non-alcoholic fatty liver disease, irritable bowel syndrome, obesity and diverse autoimmune conditions. Also notable is that there are a number of studies focusing on gut barrier dysfunction in the context of neurological conditions, although currently the outcome across these studies appear to be inconsistent.

Gut biopsy and skilled pathology examination can provide a fairly definitive indication of gut barrier dysfunction, but this is invasive and impractical. There are currently no “gold standard” non-invasive tests for gut barrier dysfunction. Non-invasive biomarkers such as serum LPS, bacterial 16S RNA and soluble CD14 are generally accepted as indicating dysfunction on a population basis. They are subject to so much variation between patients that they have no prognostic or diagnostic value for individual patients. “Sugar challenge” tests, which measure urinary excretion of e.g. lactulose and mannitol over 6-12 hours after an oral dose containing a mixture of these non-metabolised sugars, requires patients to collect this urine for subsequent mass spectroscopy, and is impractical for widespread use (with samples sent to central laboratories for analysis). At home sugar challenge tests are available on the market for home use, but the results are not well accepted clinically.

The lack of reliable biomarkers suitable for widespread use is a major barrier to: 1) better understanding the role of gut barrier dysfunction in many diseases (i.e. research use); 2) clinical development of pharmacological, dietary and other interventions to reduce gut barrier dysfunction (research use in a defined clinical setting); and 3) diagnosis of gut barrier dysfunction in those conditions where, in the clinic, it is generally accepted to be a problem (such as cirrhosis, post-surgery, burns, sepsis, irritable bowel disease).

Not only is there a paucity of markers for detecting gut barrier dysfunction there is a paucity of markers for detecting cirrhosis. Early detection of cirrhosis (Child-Pugh A) can allow intervention with therapies or behavioural modifications to prevent and/or delay patients to progressing to more severe levels of cirrhosis (Child-Pugh B and Child-Pugh C) where transplantation may be required. This is particularly important in subjects with non-alcoholic fatty liver disease (NAFLD). NAFLD affects around 25% of the global population but is usually not diagnosed until advanced disease (NASH and Grade B cirrhosis). NAFLD is increasing along with other metabolic diseases associated with obesity, and population-based screening will be in high demand within the next few years as treatments become available.

Detection of more severe levels of cirrhosis can allow for more rapid intervention with therapies and surgery.

Thus, there is a need for improved methods for identifying, screening and monitoring gut barrier dysfunction, liver disease and/or cirrhosis in a subject, including methods and kits for identifying individuals with any of the different stages of cirrhosis (Child-Pugh A, Child-Pugh B and Child-Pugh C).

A person skilled in the art will appreciate that many devices can be programed and automated to detect differences from the thresholds as described herein.

### SUMMARY OF THE DISCLOSURE

The term “and/or”, e.g., “X and/or Y” shall be understood to mean either “X and Y” or “X or Y” and shall be taken to provide explicit support for both meanings or for either meaning. As used herein, the term “about”, unless stated to the contrary, refers to +/−10%, more preferably +/−5%, even more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. As used herein, the singular form “a”, “an” and “the” include singular and plural references unless the context indicates otherwise. Each embodiment in this specification is to be applied mutatis mutandis to every other embodiment unless expressly stated otherwise.

In an aspect, the present invention provides a method for detecting gut barrier dysfunction and/or cirrhosis in a subject, the method comprising determining the dIgA level and mIgA level in a biological sample from the subject and a ratio thereof and comparing the ratio to a threshold.

In an aspect, the present invention provides a method for detecting gut barrier dysfunction and/or cirrhosis in a subject, the method comprising determining the dIgA1 level or dIgA2 level and mIgA1 or mIgA2 level in a biological sample from the subject and a ratio thereof and comparing the ratio to a threshold.

In an aspect, the present invention provides a method for detecting gut barrier dysfunction and/or cirrhosis in a subject comprising the method as described herein.

In one aspect, a difference in the level or ratio of dimeric and monomeric IgA forms as described herein from a threshold indicates gut barrier dysfunction and/or cirrhosis in the subject or a change in the level or severity of gut barrier dysfunction and/or cirrhosis in the subject. In one embodiment, the subject is retested by the subject method, and the threshold is determined from the subjects earlier test results.

In an aspect, the present invention provides a method for detecting gut barrier dysfunction and/or cirrhosis in a subject, the method comprising determining the level of IgA2 in a biological sample from the subject, wherein an elevated level of IgA2 compared to a threshold indicates gut barrier dysfunction and/or cirrhosis in the subject.

In an aspect, the present invention provides a method of treating gut barrier dysfunction and/or cirrhosis in a subject, the method comprising determining the dIgA level and mIgA level in a biological sample from the subject and a ratio thereof and comparing the ratio to a threshold, wherein when the subject has a ratio that differs from the threshold, administering the subject a treatment for gut barrier dysfunction and/or cirrhosis.

In an aspect, the present invention provides a method of treating gut barrier dysfunction and/or cirrhosis in a subject, the method comprising determining the level of IgA2 in a biological sample from the subject, wherein when the subject has an elevated level of IgA2 compared to a threshold administering the subject a treatment for gut barrier dysfunction and/or cirrhosis.

In an aspect, the present invention provides a kit for detecting gut barrier dysfunction and/or cirrhosis in a subject comprising:
(i) an agent which binds dIgA and forms a detectable dIgA complex,
(ii) an agent which binds mIgA and forms a detectable mIgA complex, wherein (i) binds specifically with dIgA and/or (ii) binds specifically with mIgA.

In an aspect, the present invention provides a test strip for a lateral flow device comprising at least one sample loading region, wherein:
a) the strip comprises a capture portion comprising an agent which binds dIgA, and
b) the strip comprises a capture portion comprising an agent which binds mIgA, wherein a) is closer to the sample loading region than b) such that the sample contacts a) before b).

In an aspect, the present invention provides a method for detecting gut barrier dysfunction and/or cirrhosis in a subject, the method comprising determining the dIgA to mIgA ratio or the mIgA to dIgA ratio in a biological sample from a subject, wherein an elevated dIgA to mIgA ratio compared to a threshold or wherein a decreased mIgA to dIgA ratio compared to a threshold indicates gut barrier dysfunction and/or cirrhosis in a subject.

In an aspect, the present invention provides a method of treating gut barrier dysfunction and/or cirrhosis in a subject, the method comprising determining the dIgA to mIgA ratio or the mIgA to dIgA ratio in a biological sample from a subject, wherein when the subject has an elevated dIgA to mIgA ratio compared to a threshold or the subject has a decreased mIgA to dIgA ratio compared to a threshold, administering the subject a treatment for gut barrier dysfunction and/or cirrhosis.

In an aspect, the present invention provides a method of treating gut barrier dysfunction and/or cirrhosis in a subject, the method comprising determining the level of IgA2 in a biological sample from the subject, wherein when the subject has an elevated level of IgA2 compared to a threshold administering the subject a treatment for gut barrier dysfunction and/or cirrhosis.

In an aspect, the present invention provides a method for detecting gut barrier dysfunction and/or cirrhosis in a subject, the method comprising;
(i) obtaining a biological sample comprising antibodies,
(ii) determining the dimeric and polymeric IgA (dIgA) level in the sample,
(iii) determining the monomeric IgA (mIgA) level in the sample,
(iv) optionally determining the IgA2 level in the sample,
(v) determining the dIgA to mIgA ratio in the sample from (ii) and (iii), wherein an elevated dIgA to mIgA ratio compared to a control and/or an elevated IgA2 level compared to a control indicates gut barrier dysfunction and/or cirrhosis in a subject.

### BRIEF DESCRIPTION OF THE FIGS.

1 A) Shows different IgA forms and their % in the blood. B) Shows an embodiment of the method of the present invention: Protein L can be used to form a complex comprising monomeric IgA; Mouse anti-human IgA2 can be used to form a complex comprising IgA2; Recombinant Chimeric Secretory Component can be used to form a complex comprising dimeric IgA. The complexes can be visualized by the addition of anti-human IgA colloidal gold and measurement of absorbance on an Axxin AX-2X reader (Runtime: 30 minutes or less). In some embodiments of the invention only dIgA and mIgA are assessed. In some embodiments of the invention dIgA, IgA2 and mIgA are assessed. In an embodiment of the invention the dIgA complex is formed before the mIgA complex. In an embodiment of the invention, the IgA2 complex is formed before the mIgA complex.

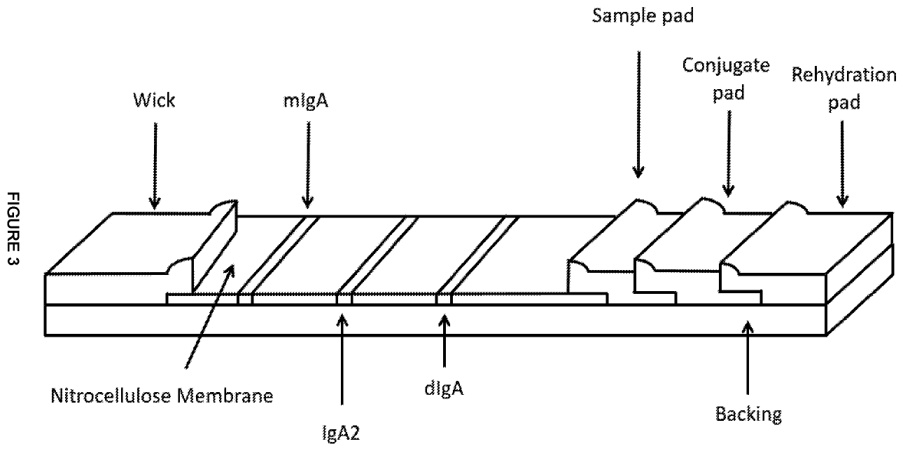

FIG. 2 Shows a diagrammatic representation, including the dimensions, of an example a lateral flow test used in Examples 2 and 3.

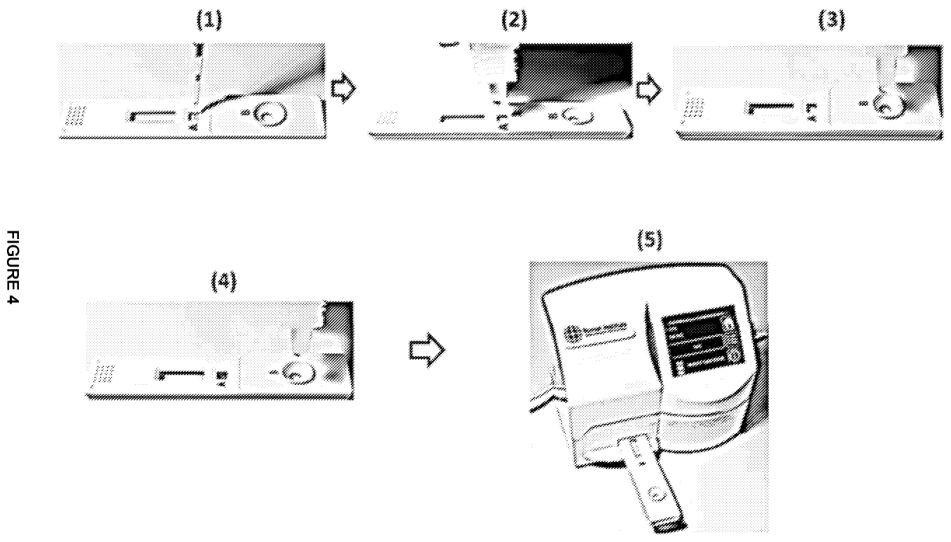

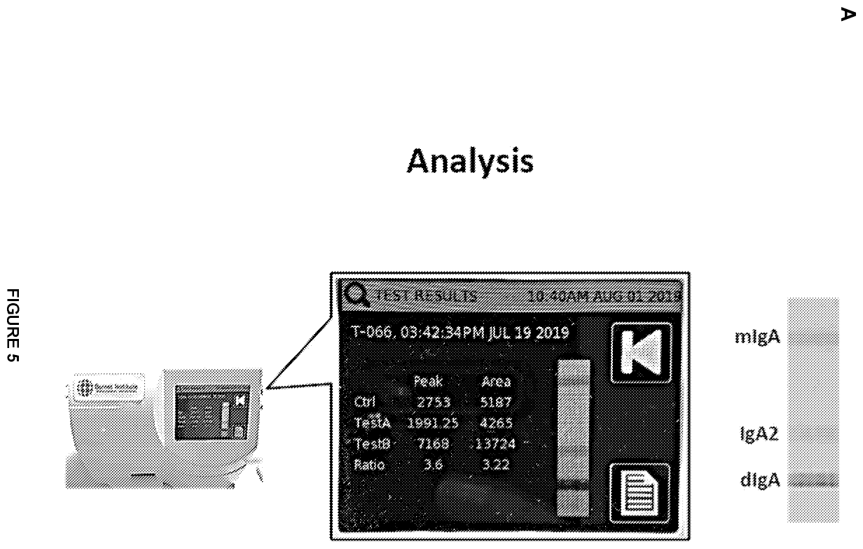

FIG. 3 Shows a diagrammatic representation, including the dimensions, of an example of a lateral flow test used in Examples 2 and 3.

FIG. 4 Illustrated the steps of one embodiment of a POC test for dIgA, IgA2 and mIgA. Plasma (5 μl) is added to well A of the cassette. Running buffer (1 drop) is added to well A and incubated for 10 mins. Four drops of running buffer is then added to well B. The test is run for 20-30 mins and read on an Axxin AX-2X reader.

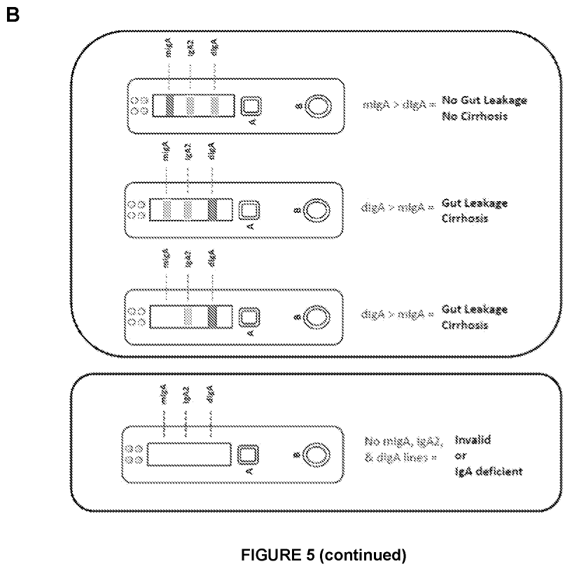

FIG. 5A) Shows an example read out of the POC test of FIG. 2. B) Shows results of running the test strip for 20 minutes (30 minutes total assay time). The test strip should show 3 visible lines, representing (in order from the sample well) dimeric IgA, IgA2, and monomeric IgA. If the third line (monomeric IgA) is missing, or if all three lines are missing, it indicates either failure of the assay, or that the patient is IgA deficient and the test cannot be used in these rare individuals. The intensity of each of the test lines is proportional to the amount of the respective analyte, and is interpreted either visually, or more preferably using an automated reader such as the Axxin AX-2X reader (Axxin Ltd, Melbourne). Using the AX-2X, a numerical readout for each test line is obtained. The workflow for using the Axxin AX-2X is shown in FIG. 5A and an example of the readout is provided in FIG. 5B.

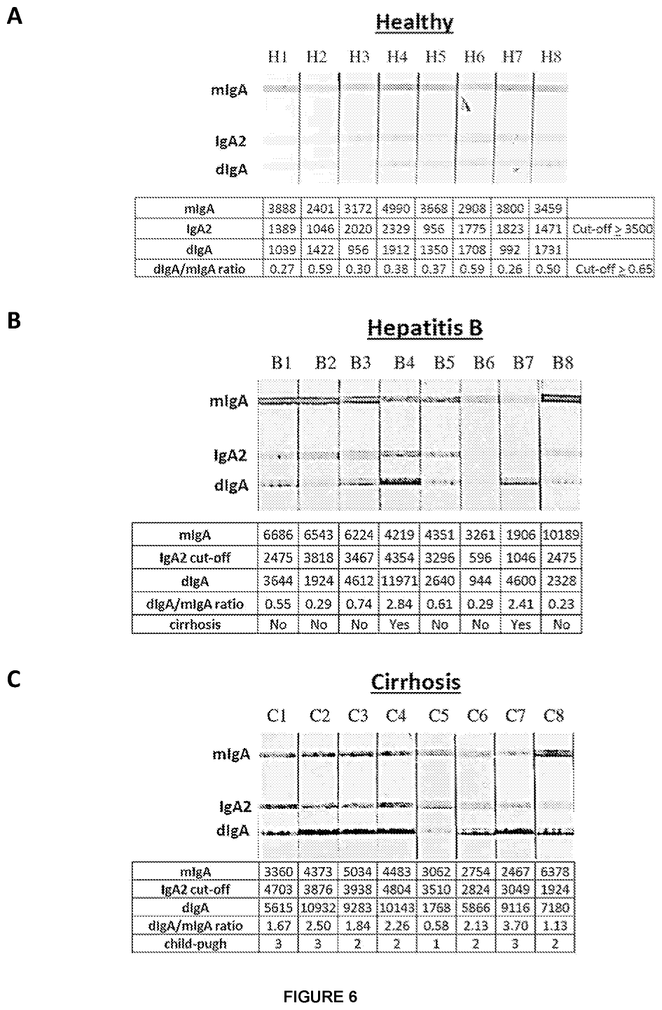

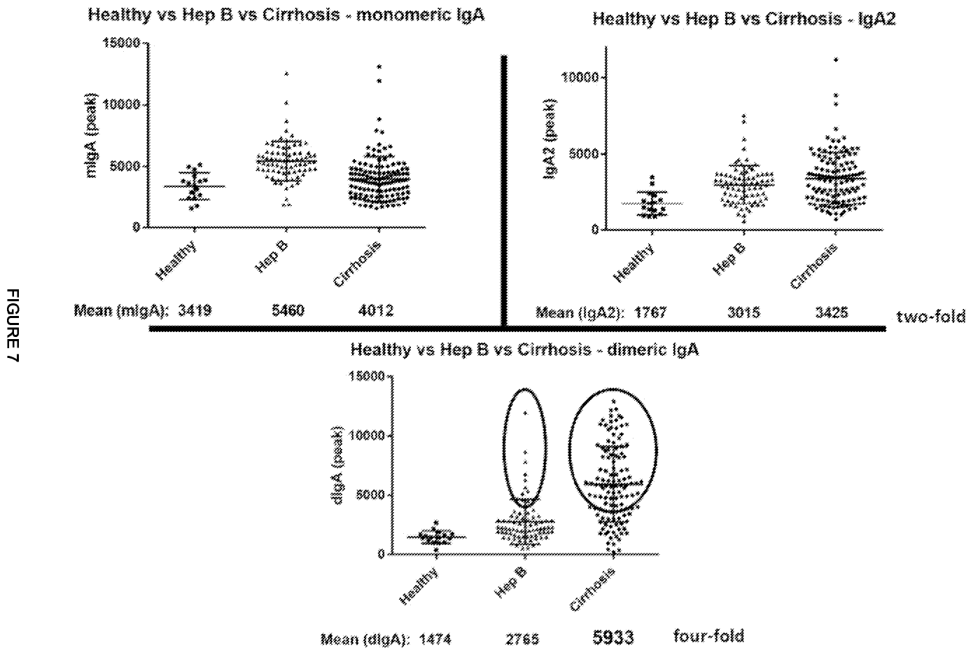

FIG. 6 Shows representative sample strips of a POC test for dIgA, IgA2 and mIgA. The POC test for dIgA, IgA2 and mIgA was performed on 16 healthy controls, 77 patients with hepatitis B virus infection, and 121 samples from cirrhotic patients. Cut-offs were established for the level of IgA2 (mean plus two standard deviations of healthy controls (≥3500)) and dIgA/mIgA ratio (mean plus 1 SD of the ratio for healthy controls (≥0.65)).

FIG. 7 Shows the assessment of dIgA, IgA2 and mIgA, in healthy individuals (n=16) compared to individuals with hepatitis B (n=77) or cirrhosis (n=121). For the individual IgA species, mIgA was elevated in chronic hepatitis B but not in cirrhotic patients, while IgA2 was elevated in both hepatitis B and cirrhotic patients, with some patients showing highly elevated levels, but the dIgA level showed the largest difference, being highly elevated in cirrhosis and in a proportion of hepatitis B patients (only 16/77 HBV have cirrhosis).

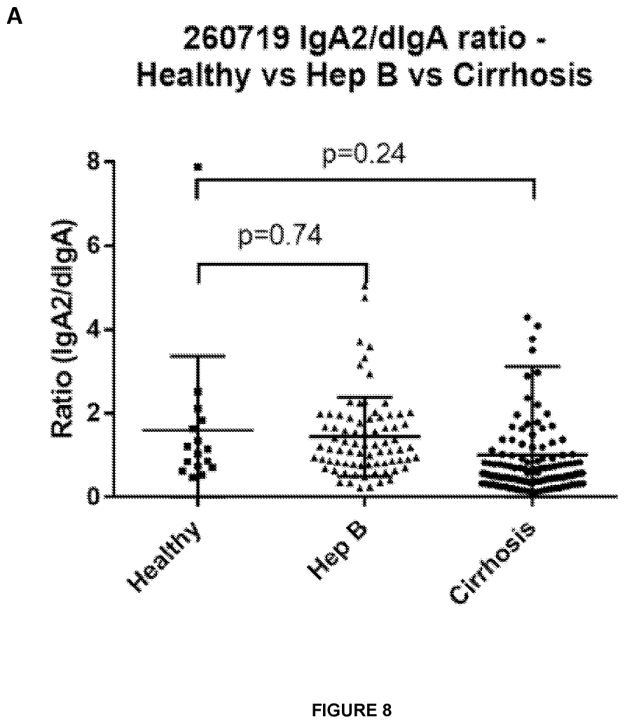

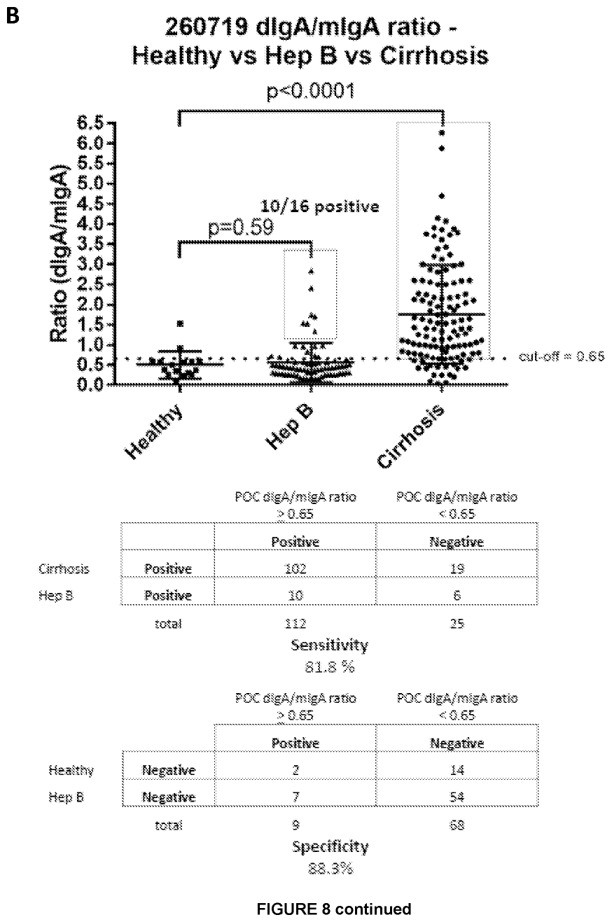

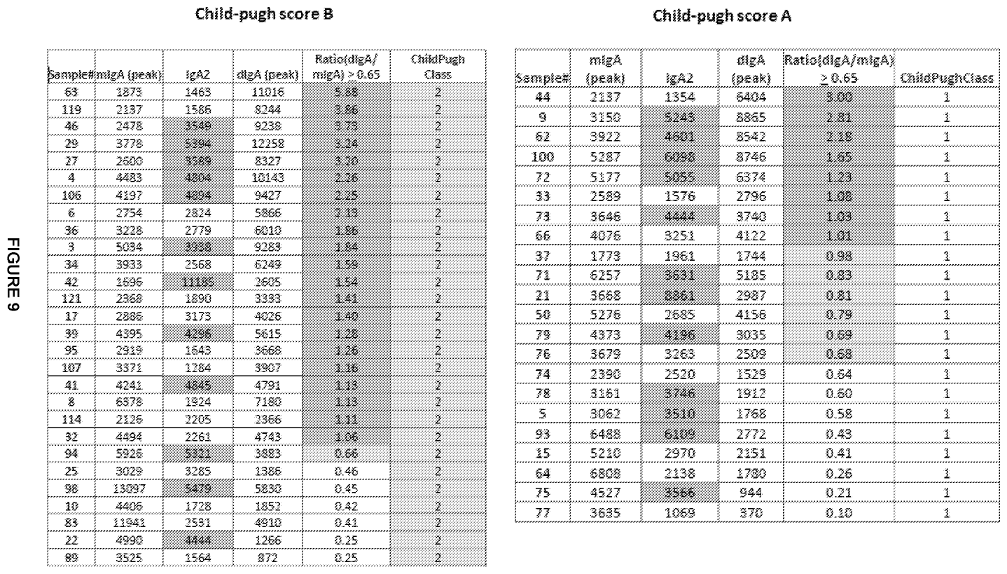

FIG. 8 Shows the ratio of IgA2/dIgA and dIgA/mIgA in healthy individuals, individuals with hepatitis B or individuals with cirrhosis. Ratios of IgA2/dIgA, or IgA2/mIgA were not significant, but the dIgA/mIgA ratio gave highly significant differences for cirrhosis versus healthy controls, and for 10/16hepatitis B patients with cirrhosis, with an overall sensitivity of 81.8% and specificity of 88.3%

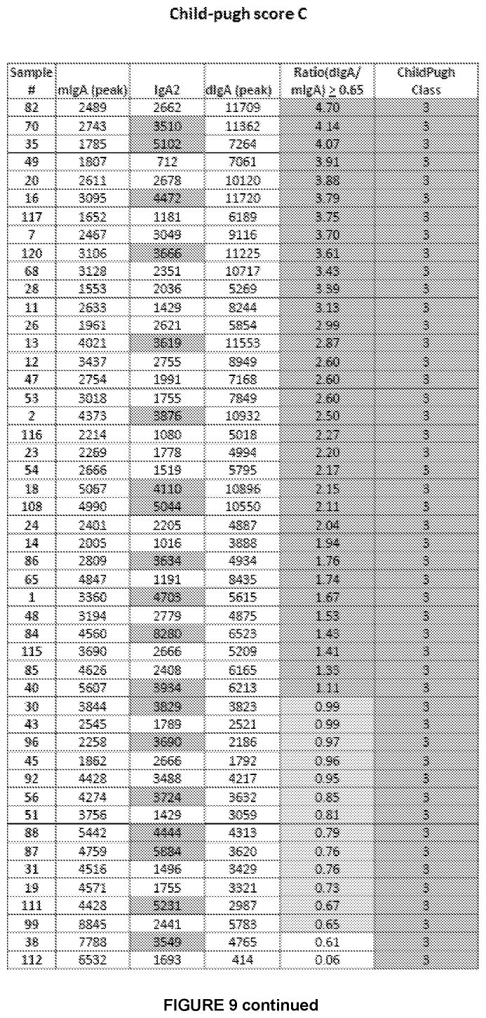

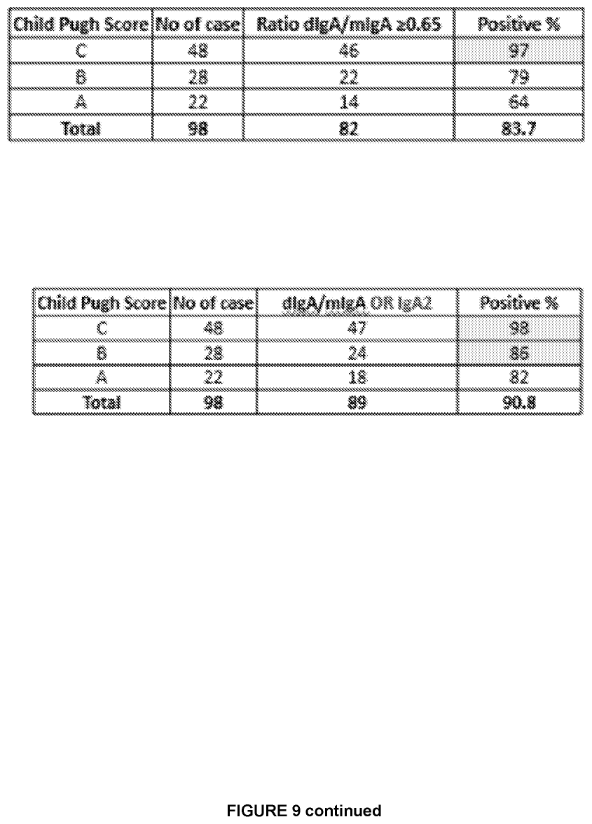

FIG. 9 Shows analysis of the dIgA/mIgA ratio with Child-Pugh cirrhosis classification. The cirrhotic patients (after excluding multiple time points for individual patients) were further examined according to the severity of cirrhosis according to the Child-Pugh classification, where C-P A is least severe and has good 1-year survival, C-P B is more severe and has moderate 1-year survival, and C-P C is most severe and has poor 1-year survival. The ratio of dIgA/mIgA was most sensitive in C-P C, and progressively less sensitive in C-P B and C-P A. However by including elevated levels of IgA2, progressively more additional cirrhotic patients were identified among the patients in each C-P class, with an overall improvement in sensitivity from 83.7% to 90.8%.

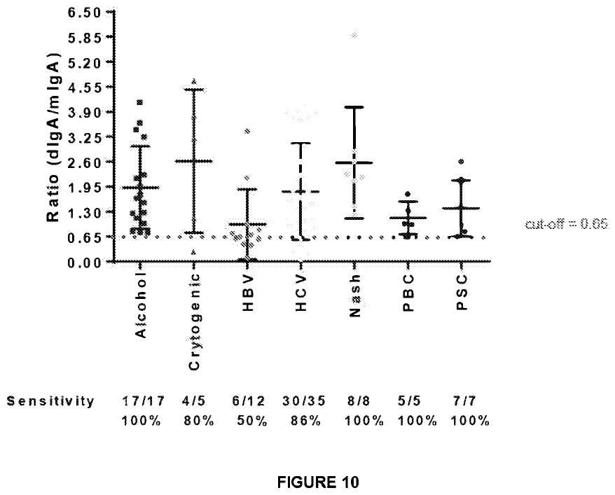

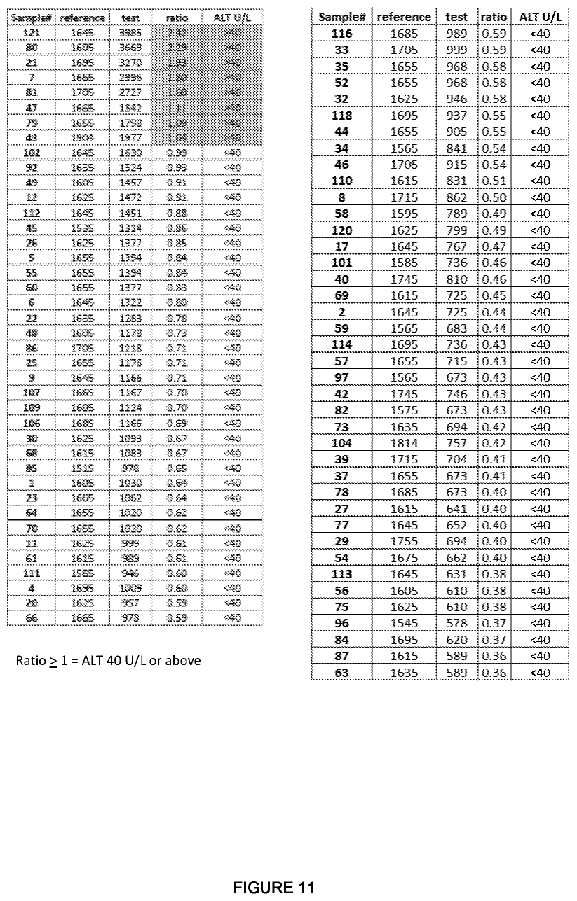

FIG. 10 Shows that most of the patients with mild cirrhosis (C-P A) who were negative on the test for dIgA/mIgA ratio were patients where the primary indication for cirrhosis was HBV infection. All other causes of cirrhosis (alcohol, cryptogenic, HCV, NASH, HIV, primary biliary cirrhosis, and primary sclerosing cholangitis) had high rates of detection, although numbers were small for some causes reflecting their lower incidence in the community. The IgA test (dIgA/mIgA ratio) had good sensitivity for cirrhosis where the primary cause was alcoholic, cryptogenic, HCV, NASH, primary biliary cirrhosis, and primary sclerosing cholangitis.

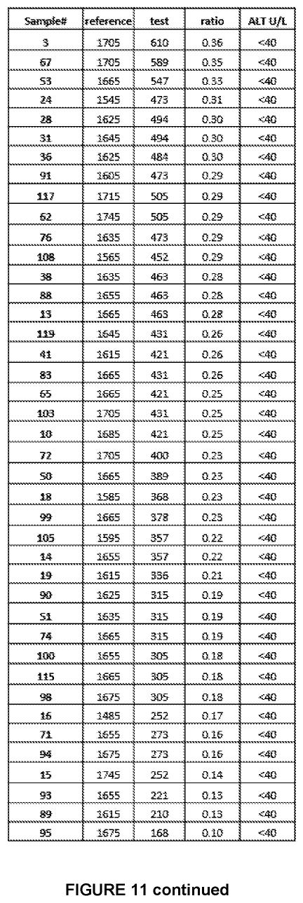

FIG. 11 Shows that when testing the same cohort as shown in FIGS. 7 to 9 for alanine aminotransferase 1(ALT-1) using the BioPoint ALT1 rapid test only a small proportion (6.6%) of cirrhotic patients have evidence of liver disease when tested using ALT, the most commonly used biomarker for liver disease. Of cirrhosis samples ( 8/121) had an ALT-1 level greater than 40 U/L. The dIgA/mIgA test complements biochemical or immunochemical tests for liver function such as the BioPoint ALT1 test in detecting more severe liver disease.

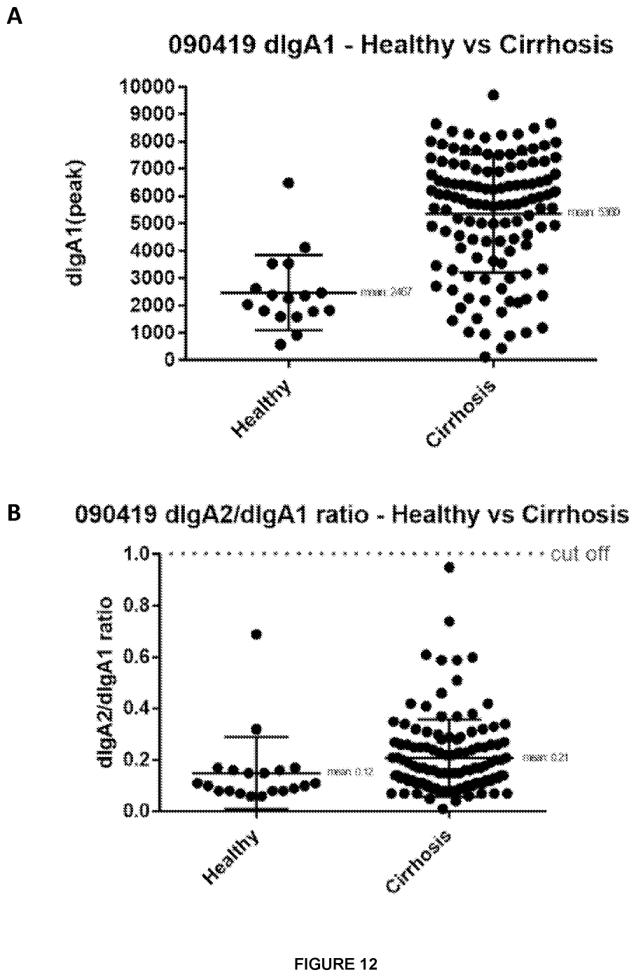

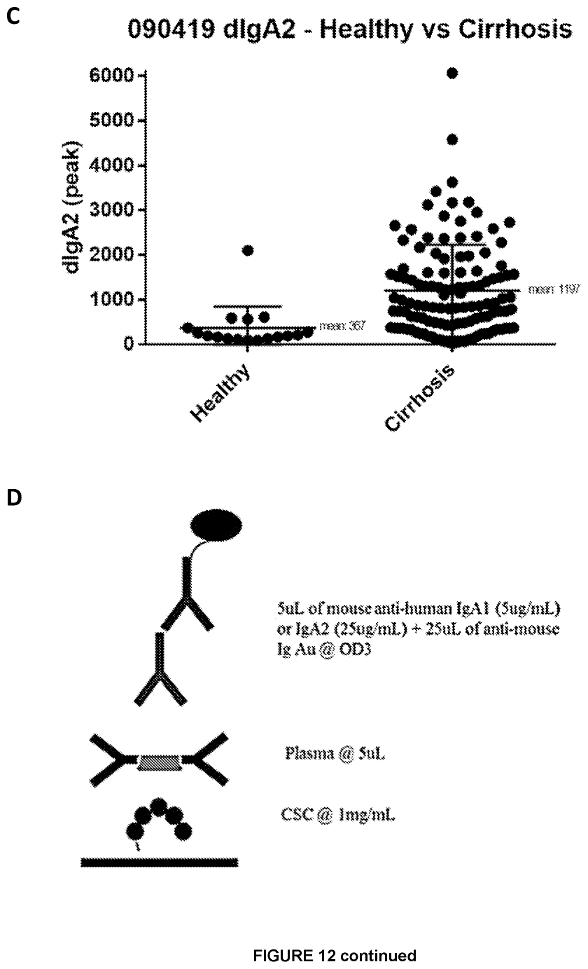

FIG. 12 Shows the separate analysis of A) dIgA1 B) dIgA2 and the C) ratio thereof in healthy (n=17) vs cirrhotic subjects (n=121). D) Provides a diagrammatic representation of the assay for detection of dIgA1 and dIgA2. In brief, dIgA1 and dIgA2 are bound to chimeric secretory component (CSC) bound to a solid support and treated with 5 μL of mouse anti-human IgA1 or mouse anti-human IgA2 which are detected by the addition of anti-mouse IgG gold. The graphs demonstrate that the level of both subclasses of dIgA (dIgA1 and dIgA2) can be measured and that both subclasses are elevated in cirrhosis. The dIgA2/dIgA1 ratios of healthy and cirrhosis samples were all below 1.

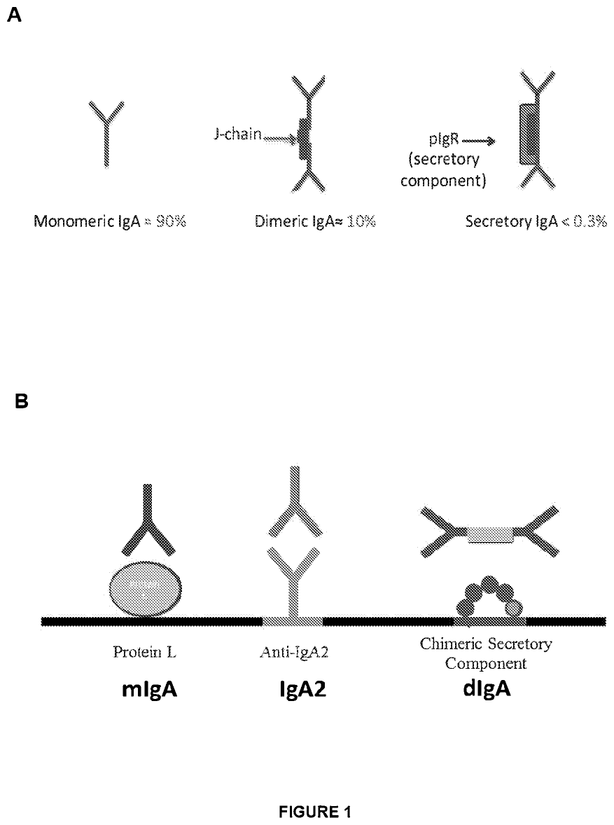

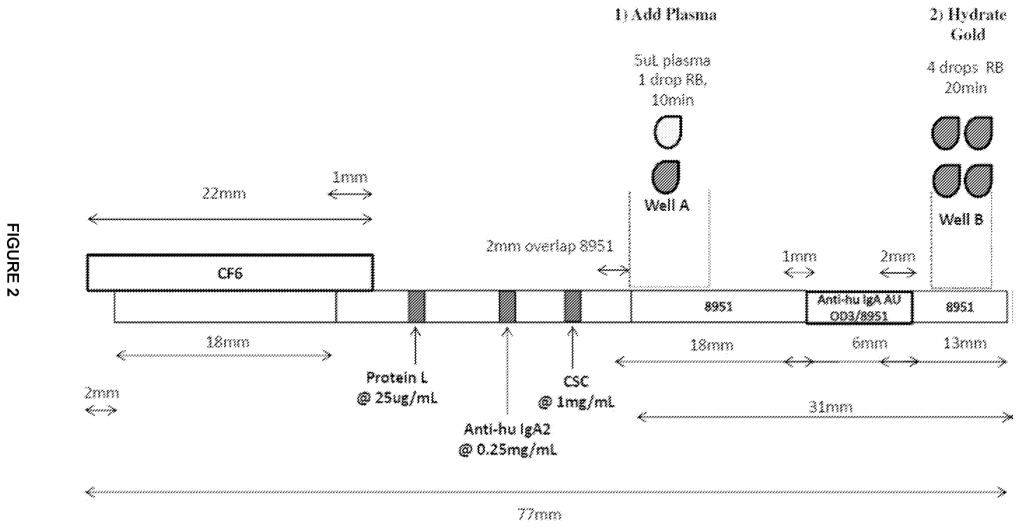

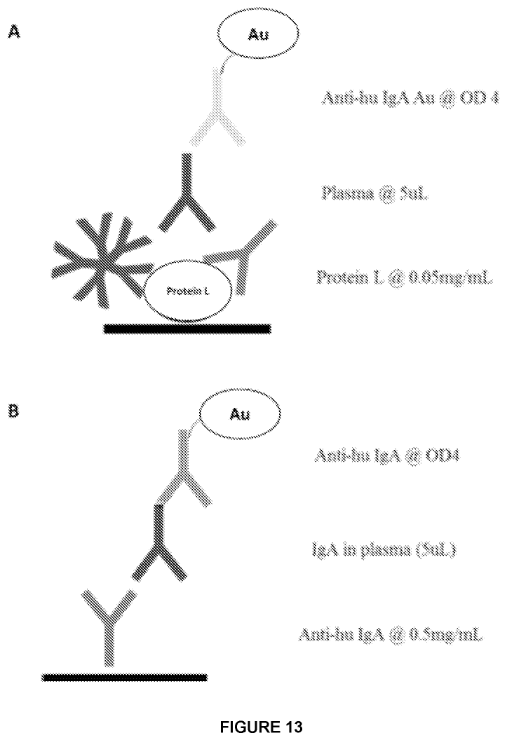

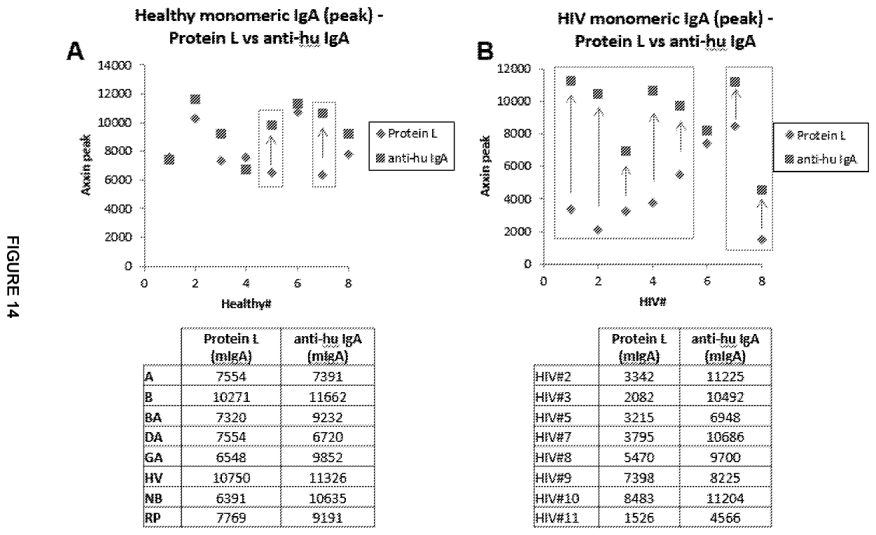

FIG. 13 Provides a diagrammatic representation of two capture methods for detecting mIgA in biological samples in the method comprising detection of dIgA, IgA2 and mIgA shown in FIG. 1B. A) For detection using protein L, Protein L is bound to a solid support, mIgA in the plasma binds protein L and is visualised by the addition of anti-human IgA gold and measurement of absorbance on an Axxin AX-2X reader. B) For detection using anti-human IgA (an antibody that detects total IgA), anti-human IgA is bound to a solid support, mIgA in the plasma is bound by the anti-human IgA and the complex is visualised by the addition of anti-human IgA gold and measurement of absorbance on an Axxin AX-2X reader.

FIG. 14 Provides a comparison of Protein L versus anti-IgA to capture mIgA for the mIgA line in the method comprising detection of dIgA, IgA2 and mIgA shown in FIG. 1B. A) Shows that in healthy controls (n=8), largely similar results are obtained for the majority of patients ( 6/8) using Protein L or anti-IgA to capture mIgA. B) Shows that in HIV patients (n=8) much larger amounts of mIgA are detected using anti-IgA capture, presumably because of competition for protein L binding by the increased overall immunoglobulin concentrations that are commonly seen during HIV infection due to generalised immune activation.

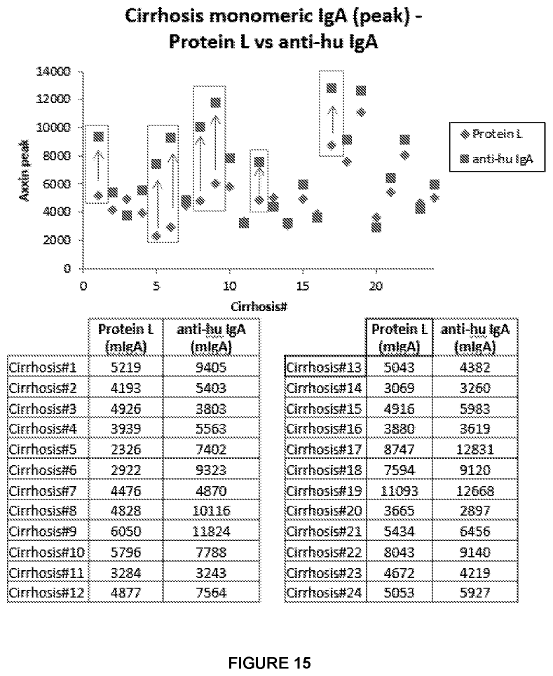

FIG. 15 Provides a comparison of Protein L versus anti-IgA to capture mIgA for the mIgA line in the method comprising detection of dIgA, IgA2 and mIgA shown in FIG. 1B in subjects with cirrhosis. The results show that for cirrhotic patients, 17/24 had similar values for mIgA using either Protein L or anti-IgA, suggesting that anti-IgA can be used for detecting mIgA in the assay format descripted in FIG. 1B.

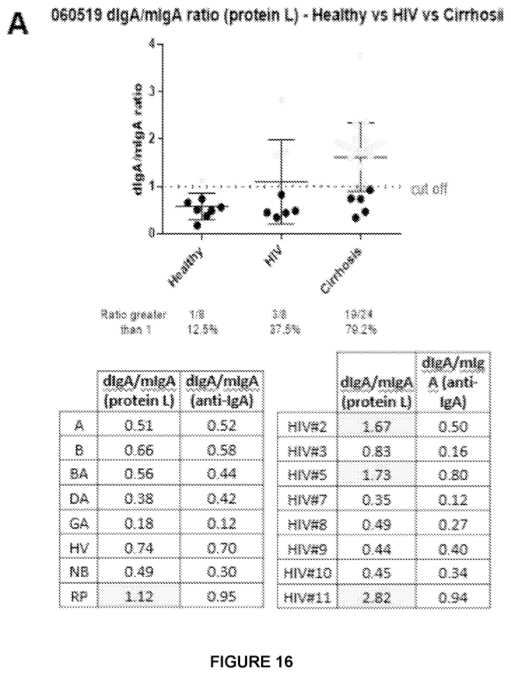

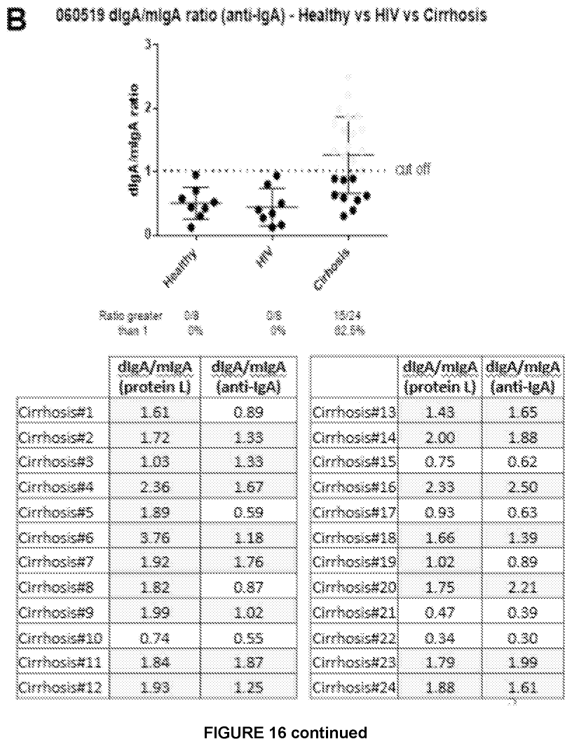

FIG. 16 Shows that using anti-IgA rather than Protein L in the dIgA/mIgA ratio test results in higher assay specificity in the selected healthy controls and HIV patients, with none of the healthy or HIV patients having a ratio above the cut-off. The cut-off used for patients and controls in this experiment was 1. However there was reduced sensitivity in the selected cirrhotic patients, with 15/24 positive (ratio>1.0) using anti-IgA to capture mIgA, whereas 19/24 were positive using Protein L to capture mIgA.

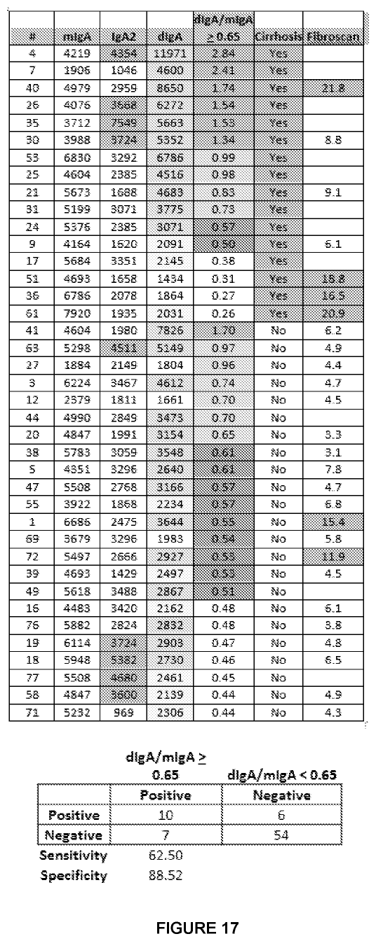

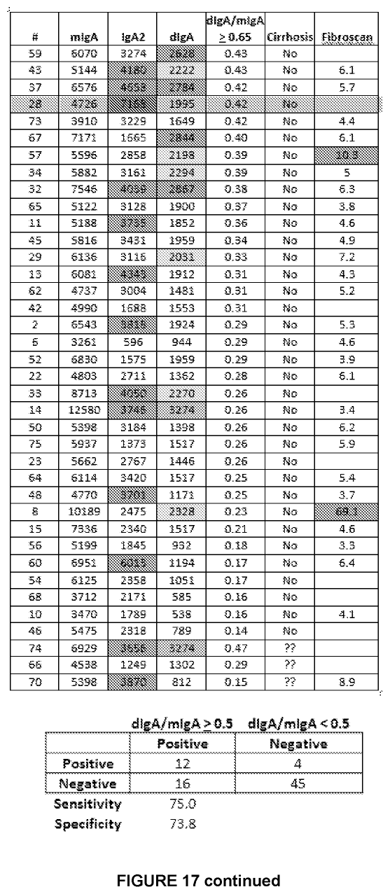

FIG. 17 shows that using a threshold of ≥0.5 for assessing for cirrhosis in a cohort of hepatitis B patients increases the sensitivity. The left panel shows the assessment of hepatitis B subjects using a threshold of ≥0.65 (which was based on mean+SD of healthy controls), a 62.5% sensitivity and a 88.5% specificity is achieved. Reducing the threshold to 0.50 increases the sensitivity to 75.0%, while specificity is still 73.8% (samples between 0.5 and 0.65 highlighted in blue). For assessment of cirrhosis in the hepatitis B patient population, sensitivity is probably more important than specificity as the prevalence of cirrhosis is 20.8% in this hepatitis B cohort, and much higher in hepatitis B cohorts in many populations such as in Africa and Asia. As such, the test can be tailored to suit for use in populations where the infection status for conditions such as hepatitis B or HIV is known.

### Discussion of Embodiments

Unless defined otherwise, all technical and scientific terms used herein have the same meanings as commonly understood by one of ordinary skill in the art to which this disclosure belongs. Any materials and methods similar or equivalent to those described herein can be used to practice or test the present disclosure. Practitioners are particularly directed to and Ausubel et al., Current Protocols in Molecular Biology, Supplement 47, John Wiley & Sons, New York, 1999; Colowick and Kaplan, eds., Methods In Enzymology, Academic Press, Inc.; Weir and Blackwell, eds., Handbook of Experimental Immunology, Vols. I-IV, Blackwell Scientific Publications, 1986; for definitions and terms of the art and other methods known to the person skilled in the art.

The detection of specific antibody (immunoglobulin (Ig)) classes is recognized as an important step in diagnostic and research methods for human and animal diseases. For example, detection of antigen-specific IgM-class antibodies is widely used as a diagnostic test for infection with viruses such as hepatitis A virus, hepatitis E virus, West Nile virus, dengue viruses, measles virus, rubella virus; and for infection with bacteria such as syphilis (Treponema pallidum), because IgM class antibodies are typically made in the body of an infected host during the acute phase of infection and are detectable for only a few months.

Conversely, IgG-class antibodies commonly persist for life and may indicate either current or past infection with a specific agent. For chronic infections such as the human immunodeficiency virus (HIV) where patients do not spontaneously clear the virus, detection of IgG-class antibodies is diagnostic for infection, whereas for others such as hepatitis C virus (HCV) where a proportion of patients do clear the virus either spontaneously or following treatment, the detection of antigen-specific IgG is not diagnostic of current or ongoing infection. IgG-class antibodies are also primarily responsible for antibody-mediated immunity within the plasma compartment of the body.

IgA-class antibodies have also been used to aid diagnosis of infections including hepatitis E virus, hepatitis A virus, and dengue viruses, as well as in the study of vaccines and immunity to infections. IgA is attractive for diagnostic purposes, because it is predominantly made during the acute phase of infection, and high levels of antigen-specific IgA can provide a marker of current infection, with or without the concurrent detection of IgM. In addition, because IgA is the predominant antibody class that is secreted at mucosal epithelial surfaces, its presence is considered as a marker of mucosal immunity. The role of different IgA structural forms as biomarkers for infection, such as specifically dIgA, is not well understood.

The term “IgA” or “total IgA” as used herein refers collectively to both subclasses (IgA1 or IgA2) and subtypes “m” “d” or “s” of IgA (overall there are six subtypes dIgA, mIgA, sIgA, dIgA2, mIgA2 and sIgA2 which fall within the two subclasses).

The term “dIgA” as used herein refers to dimeric or higher polymeric forms of IgA which are bound together by a IgA J-chain. A representative diagram of the structure is shown in FIG. 1A. The term “mIgA” as used herein refers to monomeric IgA.

The term “sIgA” as used herein refers to secretory IgA (dimeric or higher polymeric forms of IgA which have bound with the polymeric Ig receptor (pIgR) and subsequently released from the cell after cleavage of pIgR to secretory component). The interaction with pIgR is dependent on the presence of the J-chain. A representative diagram of the interaction is shown in FIG. 1A.

The term “IgA2” as used herein refers to isotype 2 of IgA. In some embodiments, the term refers to monomeric IgA2 (mIgA2). In some embodiments, dimeric and/or higher polymeric forms (dIgA2) may also be present. In one embodiment, 80% or less of the dIgA2, or 70% or less of the dIgA2, or 60% or less of the dIgA2, or 50% or less of the dIgA2, or 40% or less of the dIgA2, or 30% or less of the dIgA2, or 20% or less of the dIgA2 in a sample is present after prior reaction with a binding agent that detect dIgA such as CSC.

An IgA antibody (a total IgA antibody) can be used to detect all forms of IgA. In the methods and kits described herein an IgA antibody may be used to detect the remaining fraction of IgA after other fractions of the pool of IgA have been removed, complexed or detected via other means (e.g. via another antibody that targets a particular fraction of the IgA pool such as an antibody that targets IgA2). For example, an IgA antibody can be used to detect mIgA after dIgA and IgA2 have been complexed with antibodies or other molecules such as the chimeric secretory component (CSC) specific for dIgA and IgA2, respectively.

As used here “a normal level of alanine aminotransferase 1 (ALT-1) or alanine aminotransferase” refers to a subject having a negative result when tested with the ALT1 POC test (see Chinese patent application no. CN201610878590.7 “Point of care assays”).

Reference to “subject” includes humans and a wide range of mammals, higher primates or other animals including wild and domesticated animals, pets, pests and potential vehicles for emerging infectious diseases. In one embodiment, the subject is a mammal. In an embodiment, the mammal is a human In one embodiment, the subject is a higher primate. In an embodiment, the higher primate is a human, monkey or ape. In relation to subjects, the subjects may be suspected or diagnosed with having a disease, condition, infection or exposure to a factor associated with one or more of liver disease, gut barrier dysfunction, and cirrhosis. In an embodiment, the subject may have a diagnosis of a disease or condition that can result in gut barrier dysfunction and/or cirrhosis. In one embodiment, the cirrhosis is in a subject diagnosed with or suspected of having (non-alcoholic fatty liver disease) NAFLD. In one embodiment, the subject is diagnosed with or suspected of having (non-alcoholic steatohepatitis) NASH. In one embodiment, the subject is diagnosed with or suspected of having viral hepatitis. In one embodiment, the subject is diagnosed with or suspected of having hepatitis B. In one embodiment, the subject is diagnosed with or suspected of having hepatitis C. In one embodiment, the subject is diagnosed with hepatitis D (in addition to being diagnosed with hepatitis B). In one embodiment, the subject is diagnosed with or suspected of having HIV.

The term “gut barrier dysfunction” also referred to as “leaky gut”, “increased intestinal permeability” or “leaky gut syndrome” refers to a digestive condition were the permeability of the intestinal barrier is increased and components from inside the digestive tract (e.g. bacteria, toxins (e.g. LPS), proteins, amino acids) which through leak the intestinal wall and into the circulatory system. Some of the components that leak into the blood stream can ultimately accumulate in the liver and can contribute to cirrhosis. There are a number of causes/contributing factors to gut barrier dysfunction, including but not limited to, diet, microbial imbalances in the digestive tract, antibiotic exposure, bacterial infections, viral infections (e.g. HIV and hepatitis), cirrhosis, inflammatory bowel disease, Crohn's disease, cardiovascular disease, pulmonary disease, auto immune disease. In one embodiment, the gut barrier dysfunction is associated with cirrhosis. In one embodiment, the gut barrier dysfunction is associated with HIV infection. In one embodiment, the gut barrier dysfunction is associated with hepatitis.

The term “cirrhosis” also known as “liver cirrhosis” or “hepatic cirrhosis” as used herein refers a chronic disease of the liver marked by degeneration of cells, inflammation, and fibrous thickening of tissue. Cirrhosis is characterised by irreversible scarring of the liver. Subjects with cirrhosis may be treated to prevent/slow down further development of scar tissue in the liver or may be treated with a liver transplant. The cirrhosis may be caused by one or more of: alcohol, NAFLD, NASH, viral hepatitis, HIV, cryptogenic, primary biliary cirrhosis, and primary sclerosing cholangitis. In one embodiment, cirrhosis is caused by alcoholism. In one embodiment, the cirrhosis is in a subject diagnosed with NAFLD. In one embodiment, the subject is diagnosed with NASH. In one embodiment, the subject is diagnosed with hepatitis. In one embodiment, the subject is diagnosed with hepatitis B. In one embodiment, the subject is diagnosed with hepatitis C. In one embodiment, the subject is diagnosed with hepatitis D (in addition to being diagnosed with hepatitis B). In one embodiment, the subject is diagnosed with HIV. Clinically, a diagnosis of a subject as having cirrhosis is an indication for priority treatment (e.g. in hepatitis B subjects).

Suitable “biological sample” for the methods and kits as described herein includes any sample containing or suspected of containing antibodies that one wishes to detect including, but not limited to, biological fluids e.g. whole blood or a fraction thereof, plasma, serum or gingivo creviscular fluid. In an embodiment, the sample is whole blood. In an embodiment, the sample is any sample containing IgA antibodies. In an embodiment, the sample is a whole blood fraction, for example whole blood depleted of IgM and/or IgG. In an one embodiment, the sample is purified or partially purified. In an embodiment the sample is plasma. In an embodiment, the sample is serum. In an embodiment, the gingivo creviscular fluid. In an embodiment, biological samples are collected from a subject at two more time points. In an embodiment, the sample is obtained in close proximity to the point-of care device. In an embodiment, the sample is obtained previously for a subject. In an embodiment, the sample is stored for a period of time at about 4° C., about 15° C. or about 24° C. before use. In an embodiment, the sample is dried, freeze dried or snap frozen.

Assay Types

In an aspect, the present invention provides a method for detecting gut barrier dysfunction and/or cirrhosis in a subject, the method comprising determining the dIgA level and mIgA level in a biological sample from the subject and a ratio thereof and comparing the ratio to a threshold. In one embodiment, the method comprises detecting gut barrier dysfunction in the subject. In one embodiment, the method comprises detecting cirrhosis in the subject.

The method is suitable for in vitro use and may be performed in an assay format known to a person skilled in the art, including as an immunoassay, chromatographic assay or a homogenous assay.

As used herein, “immunoassay” refers to assays using immunoglobulins or parts thereof that are capable of detecting and quantifying a desired biomarker such as IgA and subtypes and subclasses thereof. The immunoassay may be one of a range of immune assay formats known to the skilled addressee. A wide range of immunoassay techniques are available, such as those described in Wild D. “The Immunoassay Handbook” Nature Publishing Group, 4th Edition, 2013 and subsequent innovations.

Electrochemiluminescence (ELICA), enzyme-linked immunosorbet assay (ELISA), fluorescent immunosorbent assay (FIA) and Luminex LabMAP immunoassays are examples of suitable assays to detect levels of the biomarkers. In one example, a binding agent e.g. an antibody or Protein L is attached to a support surface and a further binding reagent/antibody comprising a detectable group binds to the antibody or a substrate bound by the antibody. Examples of detectable-groups include, for example and without limitation: fluorochromes, enzymes, epitopes for binding a second binding reagent (for example, when the second binding reagent/antibody is a mouse antibody, which is detected by a fluorescently-labelled anti-mouse antibody), for example an antigen or a member of a binding pair, such as biotin. The surface may be a planar surface, such as in the case of a typical grid-type array (for example, but without limitation, 96-well plates and planar microarrays) or a non-planar surface, as with coated bead array technologies, where each “species” of bead is labelled with, for example, a fluorochrome (such as the Luminex technology described in U.S. Pat. Nos. 6,599,331, 6,592,822 and 6,268,222), or quantum dot technology (for example, as described in U.S. Pat. No. 6,306,610). Such assays may also be regarded as laboratory information management systems (LIMS):

Lateral flow assays and more recently non-lateral flow and microfluidics provide a useful set up for biological assays. Such assays can be qualitative, quantitative or semi quantitative. In microfluidic devices, small volumes of liquid are moved through microchannels generated in, for example, a chip or cartridge. A wide range of detection reagents are available including metal nanoparticles, coloured or luminescent materials. Resonance enhanced adsorption (REA) of bioconjugated metal nanoparticles offers rapid processing times and other advantages. These devices have been combined with barcode technologies to identify the patient and the analyte being tested. Computer software and hardware for assessing input data are encompassed by the present disclosure. Point-of-care devices and arrays and high throughput screening methods are also contemplated.

Qualitative assays providing an intermediate or definitive diagnosis require integrated thresholds, gates or windows that permit scoring of samples as likely or not to have a condition. Instrument readers and software are often employed to collate data and process it through a diagnostic algorithm or decision tree

In the bead-type immunoassays, the Luminex LabMAP system can be utilized. The LabMAP system incorporates polystyrene microspheres that are dyed internally with two spectrally distinct fluorochromes. Using precise ratios of these fluorochromes, an array is created consisting of different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

In one embodiment, the assay is a homogenous assay, meaning an assay format allowing the make an assay-measurement by a simple mix and read procedure without the necessity to process samples by separating or washing. Such assays do not include an immunosorbent solid phase step. In one embodiment, the homogenous assay is time-resolved Förster resonance energy transfer (FRET).

In one embodiment, the assay is a flow cytometry, bead array, lateral flow, cartridge, microfluidic or immunochromatographic based method or the like. In one embodiment, the assay is a point-of-care assay. In one embodiment, the point-of-care assay reader is an Axxin AX-2X-type reader, or equivalent or modified device. For example, the device may be modified to include LEDs and filters of the appropriate wavelength for the subject assays.

Agents for Determining the Level of dIgA, mIgA or IgA2

The agent for determining the level of IgA or a subclass or subtype thereof in the sample can be any binding agent known to a person skilled in the art that binds IgA or a subtype or subtype thereof and forms a detectable complex. The binding agent may conveniently be an antibody or an antigen-binding fragment thereof. Other suitable binding agents are known in the art and include antigen binding constructs such as binding proteins, binding peptides, affimers, affibodies, aptamers, nanobodies and mimetics.

Methods of making antigen-specific binding agents, including antibodies and their derivatives and analogs and mimetics thereof, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Florida. Aptamer technology is described for example and without limitation in U.S. Pat. Nos. 5,270,163; 5,475,096; 5,840,867 and 6,544,776. The skilled person will be able to select binding agents for use in the methods as described herein.

In one embodiment, the binding agent is an antibody or an antigen-binding fragment or derivative thereof, an antigen-binding construct such as an affimer, affibody, aptamer, nanobody and mimetics or a ligand or binding part thereof. In one embodiment, the binding agent is immobilised on a support, such as, lateral flow test strip.

Suitable binding agents for determining the dIgA level include any binding agent that specifically or non-specifically binds dIgA and forms a detectable dIgA complex. In an embodiment, the binding agent is non-specific, binding other forms of IgA which may be removed from the sample by binding with another binding agent before use with the dIgA binding agent. In one embodiment, the binding agent is specific for dIgA. In an embodiment, the binding agent is a dIgA antibody. In one embodiment, the binding agent is an anti-dIgA1 antibody, an anti-dIgA2 antibody or a combination thereof. In one embodiment, the binding agent is an antibody which binds the J-chain of dIgA (e.g. anti-IgA J chain LifeSpan Biosciences Cat no. LS-B12942 or OriGene Technologies Cat no. AM20272PU-M). In an embodiment, the binding agent is a pIgR. The term “pIgR” as used herein refers to the polymeric Ig receptor including recombinant and modified forms thereof. In some embodiments, the pIgR is a pIgR as described in the Applicants previous application WO/2014/071456. In some embodiments, the pIgR is produced in glycan deficient cells such as glycan deficient CHO cells to enhance preferential binding to dIgA over IgM. In one embodiment, pIgR is modified to bind dIgA but not substantially bind IgM by removal of human domain 1. In an embodiment, human domain 1 is replaced by rabbit domain 1. In some embodiments, the recombinant pIgR is derived from a primate such as human pIgR and comprises at least one immunoglobulin-like domain derived from a non-primate such as rabbit, mouse, rat. In some embodiments, the recombinant pIgR comprises an amino acid sequence set out in SEQ ID NO:2, or SEQ ID NO: 4, or SEQ ID NO: 6, or SEQ ID NO: 12, or SEQ ID NO: 14, or SEQ ID NO: 16 set out in WO/2014/071456 or an dIgA-binding part thereof or and a dIgA binding variant thereof. In an embodiment, the pIgR is the chimeric secretory component. The chimeric secretory component comprises rabbit domain 1 and human domains 2-5 as described in SEQ ID NO: 5 or SEQ ID NO: 6 of WO/2014/071456. In an embodiment, pIgR is conjugated to a detectable tag.

Suitable binding agents for determining the mIgA level include any binding agent that binds mIgA and forms a detectable mIgA complex. In one embodiment, the binding agent is specific for mIgA. In one embodiment, the binding agent is an anti-mIgA antibody. In an embodiment, the binding agent is an anti-IgA antibody (e.g. Mouse Anti-human IgA Sigma Cat no: 10636. In an embodiment, the binding agent is Protein L. The term “Protein L” refers to an immunoglobulin (Ig) binding protein original derived from the bacteria Peptostreptococcus magnus. Protein L can bind the kappa light chain of antibodies without interfering with the antibodies antigen binding site. It can bind all classes of Ig (IgG, IgM, IgA, IgE and IgD). The term is intended to cover modified and recombinant versions of Protein L. Protein L can be obtained, for example, from ThermoFisher Scientific (Protein L Cat no. 21189).

Suitable binding agents for determining the mIgA1 level include any binding agent that binds mIgA and forms a detectable mIgA complex. In one embodiment, the binding agent is specific for mIgA1. In one embodiment, the binding agent is an anti-mIgA1 antibody. For example, an antibody that binds to the same region of the IgA molecule that is occupied by J-chain in dIgA would be expected to be specific for mIgA over dIgA.

Suitable binding agents for determining the IgA2 level include any binding agent that binds IgA2 and forms a detectable IgA complex. In one embodiment, the binding agent is specific for IgA2. In one embodiment, the binding agent is specific for mIgA2. In one embodiment, the binding agent is an anti-human IgA2 monoclonal antibody (for example, Nordic MUbio, Cat MAHu/IgA2).

In an embodiment, an antibody as described herein may be conjugated to a detectable tag.

As used herein, the term “binds” or “binding” refers to the interaction of a binding agent e.g. a protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the antibody, will reduce the amount of labelled “A” bound to the antibody.

As used herein, the term “specifically binds” or “specific for X” shall be taken to mean a binding agent of the disclosure reacts or associates more frequently, more rapidly, with greater duration and/or with greater affinity with a particular antigen or antigens or cell expressing same than it does with alternative antigens or cells. For example, a protein that specifically binds to an antigen binds that antigen with greater affinity (e.g., 20 fold or 40 fold or 60 fold or 80 fold to 100 fold or 150 fold or 200 fold greater affinity), avidity, more readily, and/or with greater duration than it binds to other antigens, e.g., to other subclasses of IgA or to antigens commonly recognized by polyreactive natural antibodies (i.e., by naturally occurring antibodies known to bind a variety of antigens naturally found in humans). It is also understood by reading this definition that, for example, a protein that specifically binds to a first antigen may or may not specifically bind to a second antigen.

In one embodiment, the method comprises forming the dIgA complex before the mIgA complex. In one embodiment, the IgA2 complex is formed before the mIgA complex.

In one embodiment, the method comprises contacting the sample with a dIgA binding agent and forming a detectable dIgA complex, followed by contacting the sample with a mIgA binding agent and forming a detectable mIgA complex. In one embodiment, the method comprises contacting the sample with a dIgA binding agent and forming a detectable dIgA complex, followed by contacting the sample with an IgA2 binding agent and forming a detectable IgA2 complex, followed by contacting the sample with a mIgA binding agent and forming a detectable mIgA complex.

In one embodiment, the method comprises contacting the sample with a pIgR or anti-IgA J chain antibody and forming a detectable dIgA complex, followed by contacting the sample with Protein L, an anti-IgA or an anti-mIgA antibody and forming a detectable mIgA complex. In one embodiment, the method comprises contacting the sample with a pIgR or anti-IgA J chain antibody and forming a detectable dIgA complex, followed by contacting the sample with an anti-IgA2 antibody and forming a detectable IgA2 complex, followed by contacting the sample with Protein L, an anti-IgA or an anti-mIgA antibody and forming a detectable mIgA complex.

In one embodiment, the method comprises contacting the sample with a dIgA binding agent and forming a detectable dIgA complex, followed by contacting the sample with a mIgA1 binding agent and forming a detectable mIgA1 complex. In one embodiment, the method comprises contacting the sample with a dIgA binding agent and forming a detectable dIgA complex, followed by contacting the sample with an IgA2 binding agent and forming a detectable IgA2 complex, followed by contacting the sample with a mIgA1 binding agent and forming a detectable mIgA complex.

In one embedment, the detectable complexes referred to herein are directly detectable or indirectly detectable.

In one embodiment, the detectable complexes described herein are directly detectable. In one embodiment, the binding agent is conjugated to a detectable marker or microparticles comprising a detectable marker, that provide a detectable signal. In one embodiment, the detectible marker is selected from one or more of: colloidal gold, a magnetic agent, coloured latex, carboxycellulose, carbon nanoparticles and fluorescent labels. In one embodiment, the detectable marker is a visible detectable marker.

In one embodiment, the detectable complexes are indirectly detectable (can be detected by the addition of a further reagent that binds to one or more of i) the binding agent, ii) the IgA subclass or subtype bound by the binding agent, iii) the complex, and iv) a combination thereof).

In one embodiment, the reagent that detects the complex is a reagent that detects the IgA or a subclass or subtype thereof present in the complex. In an embodiment, the reagent is an antibody that detects IgA or a subclass or subtype thereof that is prepared/derived from a species that is different to the subject from which the biological sample has been obtained. For example, if the subject is a human, the reagent detects human IgA or a subclass or subtype thereof (e.g. an anti-human IgA antibody that is prepared/derived from another species e.g. a mouse, goat, rabbit, donkey etc.). For example, if the subject is a monkey the reagent detects monkey IgA or a subclass or subtype thereof (e.g. an anti-monkey IgA antibody prepared/derived from another species e.g. a mouse, goat, rabbit, donkey, human etc.).

In one embodiment, the subject is a human and the reagent is selected from one or more of: anti-human IgA colloidal gold, anti-human IgA1 colloidal gold and anti-human IgA2 colloidal gold. In one embodiment, the subject is a human and the reagent is anti-human IgA colloidal gold. In one embodiment, the subject is a human and the reagent is anti-human IgA1 colloidal gold. In one embodiment, the subject is a human and the reagent is anti-human IgA2 colloidal gold.

In one embodiment, the subject is a monkey and the reagent is selected from one or more of: anti-monkey IgA colloidal gold, anti-monkey IgA1 colloidal gold and anti-monkey IgA2 colloidal gold. In one embodiment, the subject is a monkey and the reagent is anti-monkey IgA colloidal gold. In one embodiment, the subject is a monkey and the reagent is anti-monkey IgA1 colloidal gold. In one embodiment, the subject is a monkey and the reagent is anti-monkey IgA2 colloidal gold.

In one embodiment, the detectable complex comprises one or more of: colloidal gold, a magnetic agent, coloured latex, carboxycellulose, carbon nanoparticles and a fluorescent label.

Thresholds

Based on the disclosure herein a person skilled in the art will appreciate that the level of one or more of the dIgA level, mIgA level, mIgA1 level, IgA2 level, ratio of the dIgA level to the mIgA level, ratio of the mIgA level to the dIgA level, ratio of the dIgA level to the mIgAl level, and the ratio of the mIgAl level to the dIgA level, can be compared relative to a threshold to determine if a subject has gut barrier dysfunction and/or cirrhosis. In an embodiment, comparison to the threshold can be used to determine if a subject has gut barrier dysfunction. In an embodiment, comparison to the threshold can be used to determine if a subject has cirrhosis.

The term “threshold” refers to a value, range, or cut-off that must be met, exceeded or not exceeded to determine is a subject has gut barrier dysfunction and/or cirrhosis.

In one embodiment, the present application illustrates or describes the invention with “disease” thresholds, that is, the threshold met or above which a subject is assessed as having gut barrier dysfunction and/or cirrhosis or a level thereof. As the skilled person will appreciate, the levels, and/or ratios described herein in healthy or affected subjects are useful for assessing the absence of gut barrier dysfunction and/or cirrhosis or a level thereof in such subjects.

The threshold can be set relative to a control or standard processed at the same time as the standard. Alternatively, the threshold may be predetermined based on a data set produced using the specific reagents and platform for a given embodiment of the test.

In one embodiment, the threshold is a colour intensity that can be assessed visually. In one embodiment, the colour intensity can be represented as a numerical value or numerical range.

In an embodiment, the threshold is set relative to a control. The term “control” includes any sample or group of samples that can be used to establish a knowledge base of data from a subject or subjects with a known disease status.

In one embodiment, the levels of one or more of: dIgA, mIgA, mIgA1, IgA2, ratio of the dIgA level to the mIgA level, ratio of the mIgA level to the dIgA level, ratio of the dIgA level to the mIgA1 level, ratio of the mIgA1 level to the dIgA level can be compared to a threshold level in one or more populations/groups of control subjects selected from: a normal subject cohort wherein the subjects have been predetermined not to have one or more of gut barrier dysfunction, liver disease, cirrhosis, NAFLD, HASH, hepatitis B and hepatitis C; and a population recently infected or newly diagnosed with a disease or condition that causes gut barrier dysfunction, liver disease, cirrhosis, NAFLD, HASH, hepatitis B and hepatitis C. Typically a control is a group of healthy subjects.

Thresholds may be selected that provide an acceptable ability to predict gut barrier dysfunction and/or cirrhosis in a subject. In illustrative examples, receiver operating characteristic (ROC) curves are calculated by plotting the value of one or more variables versus its relative frequency in two populations (called arbitrarily “disease” and “normal”). For any of the above listed markers, a distribution of level(s) for subjects in the two populations will likely overlap. Under such conditions, a test level does not absolutely distinguish “disease” and “normal” with 100% accuracy, and the area of overlap indicates where the test cannot distinguish between groups. Accordingly, in some embodiments, a threshold or range is selected, above which (or below which) the test is considered to be “positive” and below which the test is considered to be “negative”.

In one embodiment, the threshold is set as the mean value of the control group, the mean value plus one standard deviation of the control group, the mean value plus two standard deviations of the control group, the mean value plus three standard deviations of the control group, or a preselected level in the control group. In one embodiment, the threshold is set as the mean plus one standard deviation of a control group. In one embodiment, the threshold is set as the mean plus two standard deviations of a control group. In one embodiment, the threshold is set as the mean plus three standard deviations of a control group.

Alternatively, or in addition, thresholds may be established by obtaining a level of an IgA or a subclass or subtype thereof from the same patient, to which later results may be compared. In these embodiments, the individual in effect acts as their own “control”. In addition an individual may act as a control for e.g. a low level of cirrhosis (e.g. Child-Pugh A) which will allow their progress or the effectiveness of treatment regimens to be measured overtime. In markers that increase with disease severity, an increase over time in the same patient can indicate a worsening or development of disease or risk of disease or a failure of a treatment regimen, while a decrease or maintenance of a value over time can indicate remission, inhibition of progress of a disease state or success of a treatment regimen. Various further controls will be routinely applied by the skilled artisan.

In some embodiments, different thresholds may be required for populations with different diseases/conditions and/or different stages of a disease/conditions. An internal standard reflective of the thresholds described herein may include as an internal control in the methods and kits as described herein.

In one embodiment, of the method described herein a difference from the threshold indicates gut barrier dysfunction and/or cirrhosis in the subject. In one embodiment, the difference is an elevation relative to a threshold. The term “elevated” or “elevates” or “elevated” refers to having a higher or greater level of a protein or ratio of two proteins levels compared to a threshold. In an embodiment, the protein level or ratio of two proteins is elevated, by at least 2%, or by at least 5%, or at least 10%, or at least 15%, or at least 20%, or at least 25%, or at least 30%, or at least 35%, or at least 40%, or at least 45%, or at least 50%, or at least 60%, or at least 70%, or at least 80%, or at least 90%, or at least 100%, or at least 200%, or at least 300% compared the threshold. In one embodiment, the difference is a decrease relative to a threshold. The term “decrease” or “decreases” or “decreased” refers to abolishing, decreasing or having a lower level of a protein or ratio of two protein levels compared to a threshold. In an embodiment, the protein level or ratio of two proteins is decreased is by at least 2%, or at least 5%, or at least 10%, or at least 15%, or at least 20%, or at least 25%, or at least 30%, or at least 35%, or at least 40%, or at least 45%, or at least 50%, or at least 60%, or at least 70%, or at least 80%, or at least 90%, or at least 100% compared to the threshold.

In some embodiments, the assay comprises two or more thresholds.

In one embodiment, an elevated dIgA to mIgA ratio or elevated dIgA1 to mIgA1 ratio or dIgA2 to mIgA2 ratio compared to a threshold indicates gut barrier dysfunction and/or cirrhosis in the subject. In one embodiment, a decreased mIgA to dIgA ratio or mIgA1 to dIgA1 ratio or mIgA2 to dIgA2 ratio compared to a threshold indicates gut barrier dysfunction and/or cirrhosis in the subject.

In one embodiment, a dIgA to mIgA ratio greater than or equal to a threshold of about 0.5 to 0.8 indicates gut barrier dysfunction and/or cirrhosis in the subject. In one embodiment, a dIgA to mIgA ratio greater than or equal to a threshold of about 0.6 to 0.7 indicates gut barrier dysfunction and/or cirrhosis in the subject. In one embodiment, a dIgA to mIgA ratio greater than or equal to a threshold of about 0.65 indicates gut barrier dysfunction and/or cirrhosis in the subject. In one embodiment, a dIgA to mIgA ratio greater than or equal to a threshold of about 1 indicates gut barrier dysfunction and/or cirrhosis in a subject with HIV. In one embodiment, a dIgA to mIgA ratio greater than or equal to a threshold of about 0.5 indicates gut barrier dysfunction and/or cirrhosis in a subject with hepatitis B.

In one embodiment, a mIgA to dIgA ratio of less than or equal to a threshold of about 1.2 to 2.5 indicates gut barrier dysfunction and/or cirrhosis in the subject. In one embodiment, a mIgA to dIgA ratio of less than or equal to a threshold of about 1.5 to 2.2 indicates gut barrier dysfunction and/or cirrhosis in the subject. In one embodiment, a mIgA to dIgA ratio of less than or equal to a threshold of about 1.54 indicates gut barrier dysfunction and/or cirrhosis in the subject. In one embodiment, a mIgA to dIgA ratio less than or equal to a threshold of about 1 indicates gut barrier dysfunction and/or cirrhosis in a subject with HIV, or wherein a mIgA to dIgA ratio equal to or less than a threshold of about 2 indicates gut barrier dysfunction and/or cirrhosis in a subject with hepatitis B.

In one embodiment, an elevated level of IgA2 compared to a threshold indicates gut barrier dysfunction and/or cirrhosis in the subject. In one embodiment, an IgA2 level of greater than or equal to a threshold of 3500 DA indicates gut barrier dysfunction and/or cirrhosis in the subject.

In one embodiment, when the mIgA level is elevated relative to a threshold, then the dIgA level is compared to a dIgA threshold wherein a difference from the dIgA threshold indicates gut barrier dysfunction and/or cirrhosis in the subject, and wherein when the mIgA level is decreased relative to a threshold the ratio of the dIgA level and mIgA level is determined and compared to a ratio threshold, wherein a difference from the ratio threshold indicates gut barrier dysfunction and/or cirrhosis in the subject. In an embodiment, dIgA and mIgA are dIgAl and mIgA1. In an embodiment, dIgA and mIgA are dIgA2 and mIgA2. In one embodiment, dIgA is measured by an anti-IgA antibody. In an embodiment, the mIgA threshold is 5000 DA.

As used herein “DA” is an absorbance unit provided by a device that measures absorbance such as an Axxin AX-2X reader. Based on the information provided herein, a person skilled in the art could readily determine the equivalent threshold on equivalent and similar devices. In an alternate embodiment, the results of the assay could be converted to mg/mL.

Diagnostic Sensitivity and Specificity

As used herein “diagnostic sensitivity” refers to a diagnostic methods ability to correctly identify those with the disease (true positive rate). As used herein “diagnostic specificity” refers to a diagnostic methods ability to correctly identify those without the disease (true negative rate).

In one embodiment, the method as described herein has a diagnostic sensitivity of about 50 to about 98% for gut barrier dysfunction. In one embodiment, the method has a diagnostic sensitivity of about 60 to about 95% for gut barrier dysfunction. In one embodiment, the method has a diagnostic sensitivity of about 70 to about 90% for gut barrier dysfunction. In one embodiment, the method has a diagnostic sensitivity of about 75 to about 90% for gut barrier dysfunction. In one embodiment, the method has a diagnostic sensitivity of about 75 to about 85% for gut barrier dysfunction. In one embodiment, the method as described herein has a diagnostic sensitivity of at least 85% for gut barrier dysfunction. In one embodiment, the method as described herein has a diagnostic sensitivity of at least 80% for gut barrier dysfunction.

In one embodiment, the method as described herein has a diagnostic sensitivity of about 50 to about 98% for cirrhosis. In one embodiment, the method has a diagnostic sensitivity of about 60 to about 95% for cirrhosis. In one embodiment, the method has a diagnostic sensitivity of about 70 to about 90% for cirrhosis. In one embodiment, the method has a diagnostic sensitivity of about 75 to about 90% for cirrhosis. In one embodiment, the method has a diagnostic sensitivity of about 75 to about 85% for cirrhosis. In one embodiment, the method as described herein has a diagnostic sensitivity of at least 85% for cirrhosis. In one embodiment, the method as described herein has a diagnostic sensitivity of at least 80% for cirrhosis.

In one embodiment, the method as described herein has a diagnostic specificity of about 50 to about 98% for gut barrier dysfunction. In one embodiment, the method has a diagnostic specificity of about 60 to about 95% for gut barrier dysfunction. In one embodiment, the method has a diagnostic specificity of about 70 to about 90% for gut barrier dysfunction. In one embodiment, the method has a diagnostic specificity of about 75 to about 90% for gut barrier dysfunction. In one embodiment, the method has a diagnostic specificity of about 75 to about 85% for gut barrier dysfunction. In one embodiment, the method as described herein has a diagnostic specificity of at least 85% for gut barrier dysfunction. In one embodiment, the method as described herein has a diagnostic specificity of at least 80% for gut barrier dysfunction.

In one embodiment, the method as described herein has a diagnostic specificity of about 50 to about 98% for cirrhosis. In one embodiment, the method has a diagnostic specificity of about 60 to about 95% for cirrhosis. In one embodiment, the method has a diagnostic specificity of about 70 to about 90% for cirrhosis. In one embodiment, the method has a diagnostic specificity of about 75 to about 90% for cirrhosis. In one embodiment, the method has a diagnostic specificity of about 75 to about 85% for cirrhosis. In one embodiment, the method as described herein has a diagnostic specificity of at least 85% for cirrhosis. In one embodiment, the method as described herein has a diagnostic specificity of at least 80% for cirrhosis.

In one embodiment, when the level of IgA2 is not determined, the method has one or more of: i) a diagnostic sensitivity of at least 54% for cirrhosis with a Child-Pugh score of A; ii) a diagnostic sensitivity of at least 69% for cirrhosis with a Child-Pugh score of B; and iii) a diagnostic sensitivity of at least 87% for cirrhosis with a Child-Pugh score of C.

In one embodiment, when the level of IgA2 is determined, the method has one or more of: i) a diagnostic sensitivity of at least 72% for cirrhosis with a Child-Pugh score of A; ii) a diagnostic sensitivity of at least 76% for cirrhosis with a Child-Pugh score of B; and iii) a diagnostic sensitivity of at least 88% for cirrhosis with a Child-Pugh score of C.

In one embodiment, for cirrhosis with a Child-Pugh score of A the method has one or more of: i) a diagnostic sensitivity of at least 90% for alcoholic cirrhosis; ii) a diagnostic sensitivity of at least 70% for cryptogenic cirrhosis; iii) a diagnostic sensitivity of at least 40% for hepatitis B cirrhosis; iv) a diagnostic sensitivity of at least 76% for hepatitis C cirrhosis; v) a diagnostic sensitivity of at least 90% for NASH cirrhosis; vi) a diagnostic sensitivity of at least 90% for PBC; and vii) a diagnostic sensitivity of at least 90% for PSC.

In an embodiment, the method has a combined higher sensitivity and specificity than APRI (aspartate aminotransferase—platelet ratio index) for the detection of stage F4 fibrosis of the liver.

Methods of Treatment

The present invention also relates to a method of treating gut barrier dysfunction and/or cirrhosis in a subject determined to have such a condition using a method of the invention. In an embodiment, the method is for treating gut barrier dysfunction. In an embodiment, the method is for treating cirrhosis.

In one embodiment, the treatment is selected from: surgical intervention (e.g. to remove scar tissue or a liver transplant), administration of a therapeutic, administration of a prophylactic drug, dietary intervention, exercise intervention, or probiotic or microbiotic intervention such as “fecal transplant”, based on results.

In one embodiment, the therapeutic or prophylactic is selected from one or more of: Disulfiram, Naltrexone, Acamprosate, Corticosteroids, Prednisone, Prednisone and Azathioprine, Penicillamine, Trientine, Deferoxamine, Ciprofloxacin, Norofloxacin, Ceftriaxone, Ofloxacin, Amoxicillin-clavulanate, Vitamin K, Phytonadione, Bumetanide, Furosemide, Hydrochlorothiazide, Chlorothiazide, Amiloride, Triamterene and Spironolactone.

In one embodiment, the method comprises re-testing at a later time point to determine progression and/or response to treatment.

Kits

In various related aspects, the present invention also relates to devices and kits for performing the methods described herein. Suitable kits comprise at least some, preferably all of, the reagents sufficient for performing at least one of the methods described herein.

In an aspect, the present invention provides a kit for detecting gut barrier dysfunction and/or cirrhosis in a subject comprising: (i) an agent which binds dIgA and forms a detectable dIgA complex, (ii) an agent which binds mIgA and forms a detectable mIgA complex, wherein (i) binds specifically with dIgA and/or (ii) binds specifically with mIgA.

In an aspect, the present invention provides a kit for detecting gut barrier dysfunction and/or cirrhosis in a subject comprising: (i) an agent which binds dIgA1 and forms a detectable dIgA complex, (ii) an agent which binds mIgAl and forms a detectable mIgA1 complex, wherein (i) binds specifically with dIgA and/or (ii) binds specifically with mIgA.

In an aspect, the present invention provides a kit for detecting gut barrier dysfunction and/or cirrhosis in a subject comprising: (i) an agent which binds dIgA2 and forms a detectable dIgA complex, (ii) an agent which binds mIgA2 and forms a detectable mIgA2 complex, wherein (i) binds specifically with dIgA and/or (ii) binds specifically with mIgA.

In one embodiment, the kit further comprises a agent which binds and forms a detectable IgA2 complex. In one embodiment, one or more of the agents is bound to a solid support.

In one embodiment, the kit further comprises a reagent which detects one or more of the mIgA complex, dIgA complex, and IgA2 complex. In one embodiment, the reagent is anti-human IgA colloidal gold.

In one embodiment, the kit comprises a strip, chip or cartridge for use in a lateral flow assay. In one embodiment, the kit comprises a strip, chip or cartridge for use on point-of care device.

In one embodiment, the kit comprises a test strip for a lateral flow device comprising at least one sample loading region, wherein:
a) the strip comprises a capture portion comprising an agent which binds dIgA, and
b) the strip comprises a capture portion comprising an agent which binds mIgA,

wherein a) is closer to the sample loading region than b) such that the sample contacts a) before b). In one embodiment, the strip further comprises, between a) and b), is c) a capture portion comprising an agent which binds IgA2.

A sample can flow through the device, simultaneously or subsequently a detection reagent is flowed through the device which binds IgA through the device. Detecting the detection reagent bound to IgA can be performed in a subsequent step e.g. by measuring absorbance. In an embodiment, the detection reagent: is selected from one or more of: anti-human IgA colloidal gold, anti-human IgA1 colloidal gold and anti-human IgA2 colloidal gold.

In one embodiment, the kit can be stored at about 4° C. In one embodiment, the kit can be stored at about 15° C. In one embodiment, the kit can be stored at about 23° C.

In one embodiment, the kit comprises an immunoassay test strip. In one embodiment, the immunoassay test strip comprises a sample loading portions comprising binding agents and two or more capture portions. In one embodiment, the biological sample is contacted with the binding agents by applying the sample to a sample portion of an immunoassay test strip wherein the test strip sample portion is operably connected to spaced capture portions of the test strip and whereby the components of the sample flow from the test strip sample portion to and through the test strip capture portions, and wherein one capture portion comprises a binding agent for detecting dIgA, and wherein a second capture portion comprises a binding agent for detecting mIgA, and wherein at least one of the binding agents specifically detects their target. In one embodiment, the binding agent for detecting dIgA binds specifically with dIgA. In one embodiment, the binding agent for detecting mIgA binds specifically with mIgA. In one embodiment, the sample contacts the capture portion comprising a binding agent for detecting dIgA before contacting the capture portion comprising a binding agent for detecting mIgA. In a further embodiment, the capture portion comprises a third capture portion comprising a binding agent for detecting IgA2. In one embodiment, the sample contacts the capture portion comprising a binding agent for detecting dIgA before contacting the capture portion comprising a binding agent for detecting IgA2 and contacts the capture portion comprising the binding agent for detecting IgA2 before contacting the capture portion comprising the binding agent for detecting mIgA. In one embodiment, the capture portion is a test line (for example, a test line as shown in FIG. 2 or FIG. 3).

In one embodiment, the kit is for use in performing all of part of an assay herein disclosed. In one embodiment, the disclosure enables and provides a point-of-care device capable of performing the methods disclosed and claimed herein.

### EXAMPLES

### Example 1

### Materials

Recombinant Protein L
Supplier: ThermoFisher Scientific
Cat #: 21189
Concentration: 25 ug/mL

Mouse Anti-Human IgA
Supplier: Sigma
Cat #: 10636
Concentration: 0.5 mg/ml

Mouse Anti Human IgA2 (subclass Specific)
Supplier: Nordic Mubio
Cat #: MAHu/IgA2
Concentration: 0.25 mg/mL

CSC (Chimeric Secretory Component, Chimeric Polymeric Ig Receptor)
Supplier: In-house (Burnet Institute as described in WO/2014/071456), expressed in mammalian cells
Concentration: 1 mg/mL

Goat Anti Human IgA 40 nm Gold Conjugate
Supplier: BBI Solutions
Cat #: BA.GAHA40
Concentration: OD3

Running Buffer
Plasma: PBS+0.5% Tween+0.05% Azide Whole blood: 1% Triton X-100 in PBS

Drying Conjugate Buffer (BDS, Used for Dilution of the Gold Conjugate for Drying)
20% Sucrose, 5% Trehalose, 0.25% Tween 20, 0.35% PEG, 1% BSA, 2 mM EDTA, 10 mM Borate

Patient Samples
Samples were obtained from patients in various cohorts of liver diseases including hepatitis B, hepatitis C, or other causes of liver cirrhosis at The Alfred Hospital or St Vincent's Hospital, Melbourne, as well as healthy volunteers at the Burnet Institute, and patients with HIV infection but no known diagnosis of liver disease at The Alfred Hospital. All samples were collected under appropriate informed consent.

### Example 2

### Detection of dIgA, mIgA and IgA2 for Gut Barrier Dysfunction and Cirrhosis

Lateral flow test strips were prepared by applying three test lines across a nitrocellulose membrane (NCM) (Vivid 90, Pall Corporation) using an IsoFlow™ dispenser (Imagene Technology), and lamination of the NCM together with a combined sample pad and gold conjugate pad (Glass Fiber 8951, AHLSTROM-Munksjo) and absorbent sink (CF6 absorbent pad, GE Healthcare). The test strip was assembled by laminating the NCM, sample pad, lysis pad, and absorbent sink on an adhesive plastic backing card and cutting into 5 mm test strips (BIODOT guillotine). These strips were assembled into a disposable plastic housing (Burnet Institute). Example layout and dimensions of the test trips are shown in FIG. 2 and FIG. 3.

The first test line contains 1 mg/ml of the recombinant protein, chimeric secretory component (CSC or pIgR, comprising domain 1 that binds dIgA and substantially fails to bind IgM in the sample, such as from rabbit, and domain 2-5 from human; see WO/2014/071456; produced by the Burnet Institute) and forms a pIgR-dIgA complex on the test strip when reacted with sample plasma, serum blood or gingivo creviscular fluid.

The second test line contains 0.25 mg/ml of anti-human IgA2 monoclonal antibody (Nordic MUbio, Cat MAHu/IgA2) which forms an anti-IgA2-IgA2 complex on the test strip when reacted with sample plasma, serum, blood or gingivo creviscular fluid.

The third test line is 0.025 mg/ml recombinant Protein L (ThermoFisher Scientific, Cat 21189) which forms a Protein-L:monomeric IgA complex when reacted with sample plasma, serum, blood or gingivo creviscular fluid. Notably, Protein L also binds both IgG and IgM antibody isotypes, and so the amount of monomeric IgA bound represents the relative level of IgA compared to other isotypes as well as the absolute amount of monomeric IgA.

To run an assay, 5 microlitres of plasma is added to the sample pad of the test strip, followed by 1 drop (approximately 30 microlitres) of PBS containing 0.5% Tween20 detergent. The same method is expected to work with serum. Alternatively, 5 microlitres of whole blood can be added, followed by 1 drop of PBS containing 1% Triton X-100 detergent. The sample and buffer are allowed to flow on the strip for 10 minutes.

Subsequently, 4 drops of the same buffer are added to the colloidal gold pad, which contains OD3 of Goat anti Human IgA 40 nm Gold Conjugate (BBI Solutions, UK; Cat BA.GAHA40) in BDS (20% sucrose, 5% trehalose, 0.25% Tween 20, 1% bovine serum albumin, 2 mM EDTA, 10 mM Borate pH 8.6). The rehydrated gold and buffer are allowed to flow on the test strip for 20 minutes allowing detection of complexes comprising a human IgA antibody in the first, second and third test lines.

After 20 minutes (30 minutes total assay time), the test strip should show 3 visible lines, representing (in order from the sample well) dimeric IgA, IgA2, and monomeric IgA. If the third line (monomeric IgA) is missing, or if all three lines are missing, it indicates either failure of the assay, or that the patient is IgA deficient and the test cannot be used in these rare individuals (FIG. 5A).

The intensity of each of the test lines is proportional to the amount of the respective analyte, and is interpreted either visually, or more preferably using an automated reader such as the Axxin AX-2X reader (Axxin Ltd, Melbourne). Using the AX-2X, a numerical readout for each test line is obtained. The workflow for using the Axxin AX-2X is shown in FIG. 4 and an example of the readout is provided in FIG. 5A.

The interpretation of the test is achieved by two methods:
1) The relative amount of dIgA and mIgA ie the numerical ratio of the test line intensities for dIgA/mIgA is calculated, and if this ratio is greater than 0.65 in the examples shown, it indicates a positive test result.
2) The absolute amount of IgA2 is observed, and if this value is greater than 3,500 arbitrary units (AX-2X), it indicates a positive test result.

The calculation of these cut-off/threshold values are shown in FIG. 6.

The overall test result is considered positive if either or both of these methods generate a positive test result. The test result is considered negative if both of these methods generate a negative test result. It is possible that other arithmetic formulas could be applied to the test values to provide additional information, such as the absolute amount of dIgA.

The results of this test in the assessment in healthy subjects, patients with Hepatitis B and patients with cirrhosis is shown in FIGS. 6, 7, 8 and 17. The test is suitable for identifying patients with low and high severity of cirrhosis levels (Child-Pugh A, Child-Pugh B and Child-Pugh C see FIG. 9) and patients with cirrhosis caused by different underlying disease states (see FIGS. 10 and 17). The importance of a test for cirrhosis is further exemplified when testing the cirrhosis patients for FIGS. 6, 7 and 8 for alanine aminotransferase 1 (BioPoint ALT1 rapid test). As shown in FIG. 11 and as well known in the art, only a small proportion (6.6%) of cirrhotic patients have evidence of liver disease when tested using ALT, the most commonly used biomarker for liver disease.

### Example 3

### dIgA Test for Gut Barrier Dysfunction and Cirrhosis

Lateral flow test strips were prepared by applying three test lines across a nitrocellulose membrane (NCM) (Vivid 90, Pall Corporation) using an IsoFlow™ dispenser (Imagene Technology), and lamination of the NCM together with a combined sample pad and gold conjugate pad (Glass Fiber 8951, AHLSTROM-Munksjo) and absorbent sink (CF6 absorbent pad, GE Healthcare). The test strip was assembled by laminating the NCM, sample pad, lysis pad, and absorbent sink on an adhesive plastic backing card and cutting into 5 mm test strips (BIODOT guillotine). These strips were assembled into a disposable plastic housing (Burnet Institute). Example layout and dimensions of the test trips are shown in FIG. 2 and FIG. 3.

The first test line contains 1 mg/ml of the recombinant protein, chimeric secretory component (CSC or pIgR, comprising domain 1 that binds dIgA and substantially fails to bind IgM in the sample, such as from rabbit, and domain 2-5 from human; see WO/2014/071456; produced by the Burnet Institute) and forms a pIgR-dIgA complex on the test strip when reacted with sample plasma, serum, blood or gingivo creviscular fluid.

The second test line contains 0.25 mg/ml of anti-human IgA2 monoclonal antibody (Nordic MUbio, Cat MAHu/IgA2) which forms an anti-IgA2-IgA2 complex on the test strip when reacted with sample plasma, serum, blood or gingivo creviscular fluid.

The third test line is 0.025 mg/ml recombinant Protein L (ThermoFisher Scientific, Cat no. 21189) which forms a Protein-L:monomeric IgA complex when reacted with sample plasma, serum, blood or gingivo creviscular fluid. Notably, Protein L also binds both IgG and IgM antibody isotypes, and so the amount of monomeric IgA bound represents the relative level of IgA compared to other isotypes as well as the absolute amount of monomeric IgA.

Plasma (5 microlitres) is added to the sample pad of the test strip, followed by 1 drop (approximately 30 microlitres) of PBS containing 0.5% Tween20 detergent. Serum could be used as an alternative to plasma. Alternatively, 5 microlitres of whole blood can be added, followed by 1 drop of PBS containing 1% Triton X-100 detergent. The sample and buffer are allowed to flow on the strip for 10 minutes.

Subsequently, 4 drops of the same buffer are added to the colloidal gold pad, which contains OD3 of Goat anti Human IgA 40 nm Gold Conjugate (BBI Solutions, UK; Cat no. A.GAHA40) in BDS (20% sucrose, 5% trehalose, 0.25% Tween 20, 1% bovine serum albumin, 2 mM EDTA, 10 mM Borate pH 8.6). The rehydrated gold and buffer are allowed to flow on the test strip for 20 minutes.

After 20 minutes (30 minutes total assay time), the test strip should show 3 visible lines, representing (in order from the sample well) dimeric IgA, IgA2, and monomeric IgA. If the third line (monomeric IgA) is missing, or if all three lines are missing, it indicates either failure of the assay, or that the patient is IgA deficient and the test cannot be used in these rare individuals (FIG. 5B).

The intensity of each of the test lines is proportional to the amount of the respective analyte, and is interpreted either visually, or more preferably using an automated reader such as the Axxin AX-2X reader (Axxin Ltd, Melbourne). Using the AX-2X, a numerical readout for each test line is obtained. The workflow for using the Axxin AX-2X is shown in FIG. 4 and an example of the readout is provided in FIG. 5B.

The interpretation of the test is achieved by two methods:
1) The relative amount of dIgA and mIgA ie the numerical ratio of the test line intensities for dIgA/mIgA is calculated, and if this ratio is greater than 0.65 in the examples shown, it indicates a positive test result.
2) The absolute amount of IgA2 is observed, and if this value is greater than 3,500 arbitrary units (AX-2X), it indicates a positive test result.

The calculation of these cut-off/threshold values are shown in FIG. 6.

The overall test result is considered positive if either or both of these methods generate a positive test result. The test result is considered negative if both of these methods generate a negative test result. It is possible that other arithmetic formulas could be applied to the test values to provide additional information, such as the absolute amount of dIgA.

In the above examples, the dimeric IgA was captured with CSC and detected with colloidal gold anti-IgA which detects both IgA1 and IgA2 isotypes of IgA. To determine whether it is possible to detect dIgA1 and dIgA2 separately, similar test strips were constructed with CSC capture as before, but with the use of primary antibodies to IgA1 and IgA2 (Nordic MUbio Cat #: MAHu/IgA1 at 5 μg/ml, and Cat #MAHu/IgA2 at 25 μg/ml, respectively) and colloidal gold-conjugated goat anti-mouse antibodies (BBI, OD3) (this is represented diagrammatically in FIG. 13). The reactivity of the dIgA test lines was measured using the Axxin AX-2X reader.

The results (FIG. 12) show that levels of both dIgA1 and dIgA2 are significantly elevated in cirrhosis patients relative to healthy controls, suggesting that digA1 and/or dIgA2 could be used as alternatives to total dIgA in determination of gut leakage/cirrhosis using the methods outlined. The very low dIgA2 reactivity observed in most healthy control subjects versus strong reactivity in the majority of cirrhotic patients may offer some advantages in development of test types, for example where the detection of any visible test line for dIgA2 (rather than comparison of dIgA and mIgA) may provide an indication of gut leakage/cirrhosis.

In the above examples, monomeric IgA was captured by Protein L, which also captures IgG and IgM which means that there is competition between the mIgA present in the sample, and the IgG and IgM also present in the sample. As such, the level of mIgA detected using Protein L reflects both the absolute concentration of mIgA, and the relative concentration of mIgA and IgG and IgM in the sample. This might be especially relevant in the case of HIV infection and other conditions associated with hypergammaglobulinemia (in the case of HIV infection, resulting from generalised immune activation which in turn is linked to gut leakage). As an alternative, it may be preferable to detect mIgA independently of the level of IgG and IgM in such samples.

To examine this, we constructed test strips as before, but with the use of Mouse Anti-human IgA (Sigma, Cat #: 10636; 0.5 mg/ml) compared to Protein L (0.05 mg/ml).

The results (FIG. 14) show that for healthy control samples, the use of the alternative anti-IgA capture resulted in similar mIgA signals for the majority of samples ( 6/8) but with 2/8 samples showing much higher apparent levels of mIgA when captured by anti-IgA rather than protein L, reflecting the lack of competition for anti-IgA capture in these samples. In contrast, ⅞ HIV-infected patient samples showed much higher apparent levels of mIgA when captured by anti-IgA rather than protein L, reflecting the lack of competition for anti-IgA capture in these samples where the higher level of IgG and IgM (due to HIV infection) increased the level of competition for Protein L.

When used for the cirrhosis patient samples (FIG. 15), 17/24 patients had similar apparent levels of mIgA using either anti-IgA or Protein L as capture reagents for mIgA, while 7/24 samples had much higher levels of mIgA using anti-IgA capture. This demonstrates that anti-IgA can be used be used as an alternative to Protein L for the capture of mIgA, and by inference other IgA capture reagents may also be used.

When the dIgA/mIgA ratios were compared using the anti-IgA versus Protein L capture reagents (FIG. 16; note that in this case for the purpose of comparison we have assigned an arbitrary cut-off of 1.0 for the ratio of dIgA/mIgA), it is seen that the assay specificity is improved by the use of anti-IgA capture, with ⅛ healthy controls and ⅜ HIV patients being negative in the anti-IgA assay but positive in the Protein L assay, while sensitivity is somewhat reduced by the use of anti-IgA capture, with 4/24 cirrhosis patients being negative in the anti-IgA assay but positive in the Protein L assay. As such, further modification of the assay may be made to balance the specificity and sensitivity requirements, for example by the use of both anti-IgA and Protein L in the same or separate test lines, or the use of alternative reagents for capture of mIgA.

### Example 4

### Discussion

The methods presented in Example 2 and Example 3 provide a non-invasive test (requiring only 5 μl of blood), that is highly reproducible, and provides an objective, numerical measure (using the AX-2X instrument or similar) with a positive result for cirrhosis being the detection of one or both of the dIgA/mIgA ratio and the IgA2 level being above a cut-off. The cut-off can be determined using healthy controls, mean plus 1 or 2 standard deviations. In practice, a control sample, one or more standards, or a dose response curve could be provided for comparison.

The methods described herein are suitable for the detection of cirrhosis, both in patients with diagnosed liver disease who are being monitored for progression (HBV, HCV, non-alcoholic fatty liver disease or NAFLD) and in population-wide screening for NAFLD and its more severe form, NASH. In resource-rich settings, this would allow triage of patients for more detailed investigation of cirrhosis (ultrasound, fibroscan and other radiological investigations) prior to therapeutic interventions, but in resource-poor settings, where cirrhosis is difficult to diagnose, it would allow access to antiviral therapy for HBV (where cirrhosis is an indication for priority treatment), and would allow appropriate management of antiviral therapy for HCV (where cirrhotic patients are seen by specialists, whereas non-cirrhotics are seen in general practice).

NAFLD affects around 25% of the global population but is usually not diagnosed until advanced disease (NASH and Grade B cirrhosis). NAFLD is increasing along with other metabolic diseases associated with obesity, and population-based screening will be in high demand within the next few years as treatments become available. The methods described herein could be combined with other tests such as liver disease tests to provide a more complete picture of a subjects liver health. For example the methods/test could be used in combination with an ALT ALT1 test (e.g. ALT1 test, Nanjing BioPoint) and could enhance the results of this test by picking up even earlier stages of liver disease.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Provisional Application No. 2020900194 entitled “Detecting gut barrier dysfunction and/or cirrhosis” filed on 24 Jan. 2020, the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method for detecting gut barrier dysfunction and/or cirrhosis in a subject, the method comprising determining the dIgA level and mIgA level in a biological sample from the subject and a ratio thereof and comparing the ratio to a threshold.

2. A method for detecting gut barrier dysfunction and/or cirrhosis in a subject, the method comprising determining the dIgA1 or dIgA2 level and mIgA1 or mIgA2 level in a biological sample from the subject and a ratio thereof and comparing the ratio to a threshold.

3. The method of claim 1 or claim 2, wherein a difference from the threshold indicates gut barrier dysfunction and/or cirrhosis in the subject.

4. The method of any one of claims 1 to 3, wherein an elevated dIgA to mIgA ratio or elevated dIgA1 to mIgA1 ratio or elevated dIgA2 to mIgA2 ratio compared to a threshold indicates gut barrier dysfunction and/or cirrhosis in the subject.

5. The method of any one of claims 1 to 4, wherein a decreased mIgA to dIgA ratio or decreased mIgA1 to dIgA1 ratio or decreased mIgA2 to dIgA2 ratio compared to a threshold indicates gut barrier dysfunction and/or cirrhosis in the subject.

6. The method of any one of claim 1, 3 or 4, wherein a dIgA to mIgA ratio greater than or equal to a threshold of 0.65 indicates gut barrier dysfunction and/or cirrhosis in the subject.

7. The method of any one of claim 1, 3 or 4, wherein a dIgA to mIgA ratio greater than or equal to a threshold of 1 indicates gut barrier dysfunction and/or cirrhosis in a subject with HIV, or wherein a dIgA to mIgA ratio greater than or equal to a threshold of 0.5 indicates gut barrier dysfunction and/or cirrhosis in a subject with hepatitis B.

8. The method of any one of claim 1, 3 or 5, wherein a mIgA to dIgA ratio of less than or equal to a threshold of 1.54 indicates gut barrier dysfunction and/or cirrhosis in the subject.

9. The method of any one of claim 1, 3 or 5, wherein a mIgA to dIgA ratio less than or equal to a threshold of 1 indicates gut barrier dysfunction and/or cirrhosis in a subject with HIV, or wherein a dIgA to mIgA ratio equal to or less than a threshold of 2 indicates gut barrier dysfunction and/or cirrhosis in a subject with hepatitis B.

10. The method of any one of claims 1 or 3 to 9, wherein the dIgA level in the sample is determined by contacting the sample with a pIgR, an anti-dIgA antibody or an anti-IgA J chain antibody and forming a detectable dIgA complex.

11. The method of claim 10, wherein the pIgR is chimeric secretory component.

12. The method of any one of claims 1 or 3 to 11, wherein the mIgA level in the sample is determined by contacting the sample with an anti-mIgA antibody and forming a detectable mIgA complex.

13. The method of any one of claims 2 to 5, wherein the mIgA1 level in the sample is determined by contacting the sample with an anti-mIgA1 antibody and forming a detectable mIgA complex.

14. The method of any one of claims 1 or 3 to 11, wherein the mIgA level is determined in a sample depleted of dIgA, and IgA2 and is determined by contacting the sample with Protein L or an anti-IgA and forming a detectable mIgA complex.

15. The method of any one of claim 2 to 5 or 13, wherein the mIgA1 level is determined in a sample depleted of dIgA, and IgA2 and is determined by contacting the sample with Protein L or an anti-IgA and forming a detectable mIgA complex.

16. The method of any one of claims 1 to 15, further comprising determining the level of IgA2 in the sample, wherein one or both of (i) and (ii) indicates gut barrier dysfunction and/or cirrhosis in the subject:
(i) an elevated dIgA to mIgA ratio compared to a threshold; a decreased mIgA to dIgA ratio compared to a threshold; an elevated dIgA1 to mIgA1 ratio compared to a threshold; a decreased mIgA1 to dIgA1 ratio compared to a threshold; an elevated dIgA2 to mIgA2 ratio compared to a threshold; a decreased mIgA2 to dIgA2 ratio compared to a threshold, and
(ii) an elevated IgA2 level compared to a threshold.

17. The method of claim 16, wherein an IgA2 level of greater than or equal to a threshold of 3500 DA indicates gut barrier dysfunction and/or cirrhosis in the subject.

18. The method of claim 16 or claim 17, wherein the IgA2 level in the sample is determined by contacting the sample with an anti-IgA2 antibody and forming a detectable IgA2 complex.

19. The method of any one of claims 1, or 2 to 18, wherein the method comprises contacting the sample with a pIgR, an anti-dIgA antibody, or anti-IgA J chain antibody and forming a detectable dIgA complex, followed by contacting the sample with Protein L, an anti-IgA antibody or an anti-mIgA antibody and forming a detectable mIgA complex.

20. The method of any one of claims 1, or 2 to 19, wherein the method comprises contacting the sample with a pIgR, an anti-dIgA antibody, or anti-IgA J chain antibody and forming a detectable dIgA complex, followed by contacting the sample with an anti-IgA2 antibody and forming a detectable IgA2 complex, followed by contacting the sample with Protein L, an anti-IgA antibody or an anti-mIgA antibody and forming a detectable mIgA complex.

21. The method of any one of claims 10 to 20, wherein one or more of the mIgA complex, dIgA complex, and IgA2 complex is detected by a reagent that binds IgA.

22. The method of claim 21, wherein the reagent that binds IgA is selected from one or more of: anti-human IgA colloidal gold, anti-human IgA1 colloidal gold and anti-human IgA2 colloidal gold.

23. The method of any one of claims 10 to 22, wherein the detectable complex comprises one or more of: colloidal gold, a magnetic agent, coloured latex, carboxycellulose, carbon nanoparticles and a fluorescent label.

24. The method of any one of claims 1 to 23, wherein the biological sample is selected from whole blood, plasma, serum or gingivo creviscular fluid.

25. The method of any one of claims 1 to 24, wherein the cirrhosis is the result of: alcohol, NAFLD (non-alcoholic fatty liver disease), NASH (non-alcoholic steatohepatitis), viral hepatitis, HIV, cryptogenic, primary biliary cirrhosis, and/or primary sclerosing cholangitis.

26. The method of any one of claims 1 to 25, wherein the cirrhosis has a Child-Pugh score of A, a Child-Pugh score of B or a Child-Pugh score of C.

27. The method of any one of claims 1 to 26, wherein the method has a diagnostic sensitivity of at least 80% for cirrhosis.

28. The method of any one of claims 1 to 27, wherein the method has a diagnostic specificity of at least 85% for cirrhosis.

29. The method of any one of claims 1 to 28, wherein when the level of IgA2 is not determined, the method has one or more of:
i) a diagnostic sensitivity of at least 54% for cirrhosis with a Child-Pugh score of A;
ii) a diagnostic sensitivity of at least 69% for cirrhosis with a Child-Pugh score of B; and
iii) a diagnostic sensitivity of at least 87% for cirrhosis with a Child-Pugh score of C.

30. The method of any one of claims 1 to 29, wherein when the level of IgA2 is determined, the method has one or more of:
i) a diagnostic sensitivity of at least 72% for cirrhosis with a Child-Pugh score of A;
ii) a diagnostic sensitivity of at least 76% for cirrhosis with a Child-Pugh score of B; and
iii) a diagnostic sensitivity of at least 88% for cirrhosis with a Child-Pugh score of C.

31. The method of any one of claims 1 to 30, wherein for cirrhosis with a Child-Pugh score of A the method has one or more of:
i) a diagnostic sensitivity of at least 90% for alcoholic cirrhosis;
ii) a diagnostic sensitivity of at least 70% for cryptogenic cirrhosis;
iii) a diagnostic sensitivity of at least 40% for hepatitis B cirrhosis;
iv) a diagnostic sensitivity of at least 76% for hepatitis C cirrhosis;
v) a diagnostic sensitivity of at least 90% for NASH cirrhosis;
vi) a diagnostic sensitivity of at least 90% for PBC; and
vii) a diagnostic sensitivity of at least 90% for PSC.

32. The method of any one of claims 1 to 31, wherein the method is suitable for use in a point-of-care (POC) device.

33. The method of any one of claims 1 to 32, wherein the method comprises: a chromatographic assay, enzyme-linked immunosorbent assay, fluorescent immunosorbent assay, radiological immunosorbent assay or a homogeneous assay.

34. The method of any one of claims 1 to 33, wherein the method comprises a lateral flow format.

35. The method of any one of claims 1 to 34, performed on a lateral flow device comprising a test strip comprising at least one sample loading region, wherein:
a) the strip comprises a capture portion comprising an agent which binds dIgA, and
b) the strip comprises a capture portion comprising an agent which binds mIgA, wherein a) is closer to the sample loading region than b) such that the sample contacts a) before b).

36. The method of claim 35, which further comprises, between a) and b), is c) a third section of the substrate comprising an agent which binds IgA2.

37. The method of claim 35 or claim 36 which comprises flowing the sample through the device and simultaneously or subsequently flowing a detection reagent which binds IgA through the device, and detecting the detection reagent bound to IgA.

38. The method of claim 37, wherein the detection reagent is selected from one or more of: anti-human IgA colloidal gold, anti-human IgA1 colloidal gold and anti-human IgA2 colloidal gold.

39. The method of any one of claims 1 to 38, wherein the subject is a mammal.

40. The method of claim 39, wherein the mammal is a human.

41. The method of any one of claims 1 to 40, wherein the subject has a normal level of alanine aminotransferase 1 (ALT-1) and/or alanine aminotransferase.

42. The method of any one of claims 1 to 41, wherein the method is used to monitor the progression of cirrhosis in the subject.

43. A method for detecting gut barrier dysfunction and/or cirrhosis in a subject, the method comprising determining the level of IgA2 in a biological sample from the subject, wherein an elevated level of IgA2 compared to a threshold indicates gut barrier dysfunction and/or cirrhosis in the subject.

44. The method of claim 43, wherein a IgA2 level greater than or equal to a threshold of 3500 DA indicates gut barrier dysfunction and/or cirrhosis in the subject.

45. A method for detecting gut barrier dysfunction and/or cirrhosis in a subject, the method comprising determining the level of dIgA2 in a biological sample from the subject, wherein a level of dIgA2 that differs from a threshold indicates gut barrier dysfunction and/or cirrhosis in the subject.

46. A method of treating gut barrier dysfunction and/or cirrhosis in a subject, the method comprising administering the subject a treatment for gut barrier dysfunction and/or cirrhosis, wherein the subject was determined to have a gut barrier dysfunction and/or cirrhosis using a method according to any one of claims 1 to 45 or 56 to 57.

47. A kit for detecting gut barrier dysfunction and/or cirrhosis in a subject comprising:
(i) an agent which binds dIgA and forms a detectable dIgA complex,
(ii) an agent which binds mIgA and forms a detectable mIgA complex, wherein (i) binds specifically with dIgA and/or (ii) binds specifically with mIgA.

48. The kit of claim 47, further comprising an agent which binds and forms a detectable IgA2 complex.

49. The kit of claim 47 or claim 48, further comprising a reagent which detects one or more of the mIgA complex, dIgA complex, and IgA2 complex.

50. The kit of claim 49, wherein the reagent is anti-human IgA colloidal gold.

51. The kit of any one of clams 47 to 50, wherein one or more of the agents is bound to a solid support.

52. The kit of any one of claims 47 to 51, wherein the kit comprises a strip, chip or cartridge for use in a lateral flow assay.

53. The kit of any one of claims 47 to 52, wherein the kit comprises a strip, chip or cartridge for use on point-of care device.

54. A test strip for a lateral flow device comprising at least one sample loading region, wherein
a) the strip comprises a capture portion comprising an agent which binds dIgA, and
b) the strip comprises a capture portion comprising an agent which binds mIgA, wherein a) is closer to the sample loading region than b) such that the sample contacts a) before.

55. The test strip of claim 54, wherein the strip further comprising, between a) and b), is c) a capture portion comprising an agent which binds IgA2.

56. A method for detecting gut barrier dysfunction and/or cirrhosis in a subject, the method comprising determining the dIgA level and mIgA level in a biological sample from the subject
wherein when the mIgA level is elevated relative to a threshold, then the dIgA level is compared to a dIgA threshold wherein a difference from the dIgA threshold indicates gut barrier dysfunction and/or cirrhosis in the subject, and
wherein when the mIgA level is decreased relative to a threshold, the ratio of the dIgA level and mIgA level is determined and compared to a ratio threshold, wherein a difference from the ratio threshold indicates gut barrier dysfunction and/or cirrhosis in the subject.
